SEQUENCE LISTING (20 sequences)

SEQ ID NO: 1 - protein, 21 aa, Artificial sequence
  Synthetic
NAVPNLRGDLQVLAQKVARTK

SEQ ID NO: 2 - protein, 21 aa, Artificial sequence
  Synthetic
CAVPNLRGDLQVLAQKVCRTK

SEQ ID NO: 3 - protein, 21 aa, Artificial sequence
  Synthetic
CAVPNLRGDLQVLAQKVACTK

SEQ ID NO: 4 - protein, 21 aa, Artificial sequence
  Synthetic
CAVPNLRGDLQVLAQKVARCK

SEQ ID NO: 5 - protein, 21 aa, Artificial sequence
  Synthetic
NCVPNLRGDLQVLAQKVCRTK

SEQ ID NO: 6 - protein, 21 aa, Artificial sequence
  Synthetic
NCVPNLRGDLQVLAQKVACTK

SEQ ID NO: 7 - protein, 21 aa, Artificial sequence
  Synthetic
NAVPNCRGDLQVLAQKCARTK

SEQ ID NO: 8 - protein, 21 aa, Artificial sequence
  Synthetic
CAVPNLRGDLQVLAQKVCRTK

SEQ ID NO: 9 - protein, 22 aa, Artificial sequence
  Synthetic
NCVPNLRGDLQVLAQKVCRTKA

SEQ ID NO: 10 - protein, 22 aa, Artificial sequence
  Synthetic
  MISC_FEATURE (19)..(19): wherein X as position 19 is defined as citrulline
NCVPNLRGDLQVLAQKVCXTKA

SEQ ID NO: 11 - protein, 22 aa, Artificial sequence
  Synthetic
  MISC_FEATURE (4)..(4): wherein X at position 4 is defined as 4-hydroxyproline
NCVXNLRGDLQVLAQKVCRTKA

SEQ ID NO: 12 - protein, 22 aa, Artificial sequence
  Synthetic
CAVPNLRGDLQVLAQKVCRTKA

SEQ ID NO: 13 - protein, 22 aa, Artificial sequence
  Synthetic
  MISC_FEATURE (7)..(7): wherein X at position 7 is defined as 4-aminophenylalanine
NCVPNLXGDLQVLAQKVCRTKA

SEQ ID NO: 14 - protein, 21 aa, Artificial sequence
  Synthetic
  MISC_FEATURE (7)..(7): wherein X at position 7 is defined as citrulline
NCVPNLXGDLQVLAQKVCRTK

SEQ ID NO: 15 - protein, 22 aa, Artificial sequence
  Synthetic
  MISC_FEATURE (7)..(7): wherein X at position 7 is defined as 4-guanidinophenylalanine
NCVPNLXGDLQVLAQKVCRTKA

SEQ ID NO: 16 - protein, 21 aa, Artificial sequence
  Synthetic
  MISC_FEATURE (4)..(4): wherein X at position 4 is defined as 4-hydroxyproline
NCVXNLRGDLQVLAQKVCRTK

SEQ ID NO: 17 - protein, 7 aa, Artificial sequence
  Synthetic
  MISC_FEATURE (5)..(6): wherein X at positions 5 and 6 are defined as Ala, Cys, Asp, Glu, Phe, Gly, His, Ile, Lys, Leu, Met, Asn, Pro, Gln, Arg, Ser, Thr, Val, Trp, or Tyr
RGDLXXL

SEQ ID NO: 18 - protein, 20 aa, Artificial sequence
  Synthetic
  MISC_FEATURE (1)..(1): wherein X at position 1 is defined as cysteine or asparagine
  MISC_FEATURE (2)..(2): wherein X at position 2 is defined as cysteine or  Alanine
  MISC_FEATURE (4)..(4): wherein X at position 4 is defined as proline or 4-hydroxyproline
  MISC_FEATURE (14)..(14): wherein X at position 14 is defined as alanine
  MISC_FEATURE (19)..(19): wherein X at position 19 is defined as arginine, citrulline, or cysteine
XXVXNLRGDLQVLXQKVCXT

SEQ ID NO: 19 - protein, 21 aa, Artificial sequence
  Synthetic
  MISC_FEATURE (1)..(1): wherein X at position 1 is defined as cysteine or asparagine
  MISC_FEATURE (2)..(2): wherein X at position 2 is defined as cysteine or Alanine
  MISC_FEATURE (4)..(4): wherein X at position 4 is defined as proline or 4-hydroxyproline
  MISC_FEATURE (14)..(14): wherein X at position 14 is defined as alanine
  MISC_FEATURE (19)..(19): wherein X at position 19 is defined as arginine, citrulline, or cysteine
  MISC_FEATURE (21)..(21): wherein X at position 21 is defined as lysine
XXVXNLRGDLQVLXQKVCXTX

SEQ ID NO: 20 - protein, 22 aa, Artificial sequence
  Synthetic
  MISC_FEATURE (1)..(1): wherein X at position 1 is defined as cysteine or asparagine
  MISC_FEATURE (2)..(2): wherein X at position 2 is defined as cysteine or Alanine
  MISC_FEATURE (4)..(4): wherein X at position 4 is defined as proline or 4-hydroxyproline
  MISC_FEATURE (14)..(14): wherein X at position 14 is defined as alanine
  MISC_FEATURE (19)..(19): wherein X at position 19 is defined as arginine or citrulline
  MISC_FEATURE (21)..(21): wherein X at position 21 is defined as lysine
  MISC_FEATURE (22)..(22): wherein X at position 22 is defined as alanine
XXVXNLRGDLQVLXQKVCXTXX

(19) United States
(12) Patent Application Publication
(10) Pub. No.: US 2024/0209034 A1
(43) Pub. Date: Jun. 27, 2024

(54) ENGINEERING PEPTIDES FOR A A VB6 INTEGRIN BINDING AND RELATED METHODS OF USE AND SYNTHESIS

(71) Applicants: University of Washington, Seattle, WA (US); Seattle Children's Hospital d/b/a Seattle Children's Research Institute, Seattle, WA (US)

(72) Inventors: Drew SELLERS, Seattle, WA (US); Ian CARDLE, Seattle, WA (US); Suzie Hwang PUN, Seattle, WA (US)

(21) Appl. No.: 18/554,737

(22) PCT Filed: Apr. 8, 2022
(86) PCT No.: PCT/US2022/024095

§ 371 Date: Oct. 10, 2023

Related U.S. Application Data
(60) Provisional application No. 63/173,964, filed on Apr. 12, 2021.

(51) Int. Cl. C07K 14/005; A61K 38/00

(52) U.S. Cl. CPC C07K 14/005; A61K 38/00; C12N 2770/32122; C12N 2770/32133

(57) ABSTRACT

Embodiments of the claimed invention are directed to synthetic peptides comprising an amino acid sequence of X₁X₂VX₃NLRGDLQVLX₄QKVCX₅T (SEQ ID NO:18), wherein at least one of X₁ and X₂ is a cysteine, and wherein one or more of X₃, X₄, and X₅ is a non-natural amino acid. Also described are methods of using the claimed embodiments for inhibiting growth of a cancer cell overexpressing integrin αvβ6. Additionally, also described are methods of using the claimed embodiments for detecting a cancer cell overexpressing integrin αvβ6.

### CROSS-REFERENCE TO RELATED APPLICATION

This application claims the benefit of U.S. Provisional Application No. 63/173,964 filed Apr. 12, 2021.

### STATEMENT OF GOVERNMENT LICENSE RIGHTS

This invention was made with government support under Grant Nos. R01 NS118247, R01 CA177272, and R21 NS099654, awarded by the National Institutes of Health, and Grant No. DGE1762114, awarded by the National Science Foundation. The government has certain rights in the invention.

### STATEMENT REGARDING SEQUENCE LISTING

The sequence listing associated with this application is provided in text format in lieu of a paper copy and is hereby incorporated by reference into the specification. The name of the file text containing the sequence listing is Seq_List_FINAL_20220405_ST25.txt. The text file is 9.0 KB; was created on Apr. 5, 2022; and is being submitted via EFS-Web with the filing of the specification.

### BACKGROUND

From 2009 through 2015, pancreatic, liver, lung, and esophageal cancers had the lowest survival rates of any cancer and are projected to contribute to 38% of cancer-related deaths in 2020. Patients are often asymptomatic at early stages with these cancers, preventing timely diagnosis and thereby limiting effective treatment options at later stages of disease. Consequentially, there is a significant need for targeted diagnostics and therapeutics that could identify and treat these cancers at early stages to improve patient outcomes.

Integrins are a family of heterodimeric transmembrane receptors that interact with proteins in the extracellular matrix (ECM) and on other cells to mediate cell adhesion and migration. While integrins are involved in a variety of healthy biological functions, including embryogenesis, tissue regeneration, and immune cell trafficking, their aberrant expression and activity can drive cancer initiation and metastasis. Integrins have thus garnered considerable interest as diagnostic and therapeutic targets for cancer. One such integrin, αvβ6, is an epithelial-restricted integrin involved in wound healing that has low basal expression in healthy tissue. αvβ6 is broadly upregulated in many solid tumor types, including pancreatic, liver, lung, esophageal, cervical, breast, head and neck, colon, ovarian, stomach, and oral cancers and its overexpression often correlates with a poor prognosis. The role of αvβ6 in tumorigenesis is correspondingly extensive; αvβ6 binds to fibronectin and tenascin for cell adhesion and migration, it activates pro-transforming growth factor beta to promote the epithelial-to-mesenchymal transition, and it mediates secretion of matrix metalloproteinases that remodel the ECM for cancer growth and invasion. Given these qualities, the integrin αvβ6 has become the focus of considerable research efforts in the last two decades as a potential target for cancer imaging and therapy.

Peptides are attractive targeting ligands for cancer due to their chemical synthesis and small size, enabling inexpensive production, ease of modification, and enhanced solid tumor penetration compared to antibodies. A20FMDV2 is a 20-amino acid, arginine-glycine-aspartate (RGD)-containing peptide derived from the G-H loop of the capsid protein VP1 from foot-and-mouth disease virus (FMDV) serotype O₁ that binds integrin αvβ6 with low nanomolar affinity and high specificity. With its favorable binding properties and demonstrated preclinical safety, A20FMDV2 has been used in many cancer research applications, including imaging of αvβ6⁺ tumors in mice and humans, αvβ6-specific drug delivery in vitro and in vivo, and engineering chimeric antigen receptors for αvβ6-directed adoptive T-cell immunotherapy. Recent studies also show the utility of the peptide for imaging idiopathic pulmonary fibrosis and those associated with connective tissue disease, radiation therapy, and severe acute respiratory syndrome coronavirus 2 (SARS-CoV-2) infection.

However, the clinical translation of A20FMDV2 has been limited, in part, by poor metabolic stability of the peptide that impairs its pharmacokinetics. Modification of A20FMDV2 with two short PEG chains (˜1 kDa each) reduces peptide degradation and thereby increases tumor retention, but also slows peptide clearance from healthy tissue and increases renal retention. In view of the limitation of the present art, a need remains for an engineered A20FMDV2 peptide with chemistries and amino acid modifications that increase the peptide's inherent metabolic stability, which improving its integrin specificity. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a synthetic peptide comprising an amino acid sequence of X₁X₂VX₃NLRGDLQVLX₄QKVCX₅T (SEQ ID NO:18), wherein at least one of X₁ and X₂ is a cysteine. The synthetic peptide can further comprise an amino acid sequence of X₁X₂VX₃NLRGDLQVLX₄QKVCX₅TX₆ (SEQ ID NO:19), wherein at least one of X₁ and X₂ is a cysteine. Additionally, the synthetic peptide can further comprise an amino acid sequence of X₁X₂VX₃NLRGDLQVLX₄QKVCX₅TX₆X₇ (SEQ ID NO:20), wherein at least one of X₁ and X₂ is a cysteine. In some embodiments X₁ can be a cysteine. In some embodiments, the synthetic peptide has a cyclic portion at X₁ and the second cysteine, wherein the peptide has at least one perfluoroaryl compound linker at a thiol group of X₁ and a thiol group of the second cysteine. In still other embodiments, X₂ can be a cysteine. In some embodiments, the synthetic peptide has a cyclic portion at X₂ and the second cysteine, wherein the peptide has at least one perfluoroaryl compound linker at a thiol group of X₂ and a thiol group of the second cysteine. In some embodiments, one or more of X₃, X₄, X₅, X₆, and X₇ is a non-natural amino acid. In still other embodiments, one or more of X₃, X₄, X₅, X₆, and X₇ is a non-natural amino acid independently selected from the group consisting of a hydroxyproline (Pₕ), a D-arginine (R_{D}), a D-alanine (A_{D}), a citrulline (Cit), and a D-lysine (K_{D}). In still other embodiments, the synthetic peptide can further comprise a therapeutic agent bound to one or more of X₁, X₆, and X₇. In some embodiments, the synthetic peptide can further comprise an imaging agent bound to one or more of X₁, X₆, and X₇.

In another aspect, the disclosure provides a cyclized peptide that specifically binds to integrin αvβ6, the peptide comprising a sequence as set forth in one of SEQ ID NOs: 2-7. The cyclized peptide comprises a cysteine linker with at least one perfluoroaryl compound, wherein at least one perfluoroaryl compound linker creates a cyclic peptide comprising 10-18 amino acids. In still other embodiments, the cyclized peptide further comprises one or more amino acid modifications as set forth in SEQ ID NOs: 9-16. In still other embodiments, the synthetic peptide can further comprise a therapeutic agent. In some embodiments, the therapeutic agent is bound to one or more of the N-terminus or the C-terminus of the peptide. In some embodiments, the synthetic peptide can further comprise an imaging agent. In some embodiments, the imaging agent is bound to one or more of the N-terminus or the C-terminus of the peptide.

In another aspect, the disclosure provides a method of inhibiting growth of a cancer cell overexpressing integrin αvβ6. The method comprising administering to a patient in need thereof a therapeutically effective amount of a composition comprising a synthetic peptide that specifically binds αvβ6 and a pharmaceutically acceptable carrier, the synthetic peptide comprising an amino acid sequence selected from the group of X₁X₂VX₃NLRGDLQVLX₄QKVCX₅T (SEQ ID NO:18), X₁X₂VX₃NLRGDLQVLX₄QKVCX₅TX₆ (SEQ ID NO:19), and X₁X₂VX₃NLRGDLQVLX₄QKVCX₅TX₆X₇ (SEQ ID NO:20). In some embodiments, the method further comprises a therapeutic agent bound to the synthetic peptide. In still other embodiments, the synthetic peptide comprises an amino acid sequence of X₁X₂VX₃NLRGDLQVLX₄QKVCX₅TX₆ (SEQ ID NO:19), wherein the therapeutic agent is bound to X₆.

In another aspect, the disclosure provides an in vivo method of detecting a cancer cell overexpressing integrin αvβ6, the method comprising administering to a patient a composition comprising a synthetic peptide that specifically binds αvβ6 and an imaging agent, the synthetic peptide comprising an amino acid sequence selected from the group of X₁X₂VX₃NLRGDLQVLX₄QKVCX₅T (SEQ ID NO:18), X₁X₂VX₃NLRGDLQVLX₄QKVCX₅TX₆ (SEQ ID NO:19), and X₁X₂VX₃NLRGDLQVLX₄QKVCX₅TX₆X₇ (SEQ ID NO:20). In some embodiments, the synthetic peptide is bound to an in vivo imaging agent. In still other embodiments, the synthetic peptide comprises an amino acid sequence of X₁X₂VX₃NLRGDLQVLX₄QKVCX₅TX₆ (SEQ ID NO:19), wherein the in vivo imaging agent is bound to X₆.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1D. Site-specific cyclization of A20FMDV2 via perfluorarylation retains peptide binding to αvβ6⁺ cancer cells. FIG. 1A, 3D model of A20FMDV2 peptide predicted by PEP-FOLD3 computational framework (Lamiable, A., Thévenet, P., Rey, J., Vavrusa, M., Derreumaux, P., and Tufféry, P. (2016) PEP-FOLD3: faster de novo structure prediction for linear peptides in solution and in complex. Nucleic Acids Res. 44, W449-W454). Amino acids are listed, and positions substituted with cysteines for cyclization are shown in gray. FIG. 1B, Chemical structure of DFBP molecular linker used for cyclization. FIG. 1C, Amino-acid sequences of A20FMDV2 peptide and DFBP-cyclized variants. Cysteine substitutions for cyclization by perfluoroarylation are shown in gray. The RGDLXXL (SEQ ID NO:17) motif that is important for αvβ6 recognition is underlined in all sequences. FIG. 1D, Flow cytometry binding curves of A20FMDV2 peptide and DFBP-cyclized variants to K562 and K562 αvβ6:mCherry cells, normalized to 400 nM A20FMDV2 binding to K562 αvβ6:mCherry cells. The curves represent a nonlinear regression of one independent experiment in which binding data are fitted to a Hill equation. K_{D} values are not shown here and will be reported for promising peptides in a later figure with triplicate datasets. SA-AF647, streptavidin Alexa Fluor 647. A20FMD2 (SEQ ID NO:1); C1C18 (SEQ ID NO:2); C1C19 (SEQ ID NO:3); C1C20 (SEQ ID NO:4); C2C18 (SEQ ID NO:5); C2C19 (SEQ ID NO:6); and C6C17 (SEQ ID NO:7).

FIGS. 2A through 2C. Partial DFBP-cyclized A20FMDV2 candidates display moderate serum stability with exocyclic C-terminal degradation. FIGS. 2A-2C, MALDI-ToF spectra of DFBP-cyclized C1C18 (SEQ ID NO:2), C1C20 (SEQ ID NO:4), and C2C18 (SEQ ID NO:5) peptide variants incubated in normal mouse serum for 0, 2, 4, and 6 h at 37° C. Molecular weights of prominent peaks are shown. No peptide-fragment peaks were observed for C1C20 DFBP at the 4 and 6 h timepoints. Bottom: predicted amino acid sequences of degradation products based on measured molecular weights.

FIGS. 3A through 3B. Further modifications to DFBP-cyclized A20FMDV2 peptides do not impact binding αvβ6⁺ cancer cells. FIG. 3A, Schematic of modifications made to the sequences of C1C18 DFBP and C2C18 DFBP to further improve their serum stability. Chemical structures of the original (black) and modified (gray) amino acids are shown at positions of incorporation. The resulting modified peptide sequence are also listed, with cysteine substitutions for DFBP cyclization shown in gray and amino acid modifications shown in dark gray. The RGDLXXL (SEQ ID NO:17) motif that is important for αvβ6 recognition is underlined in all sequences. FIG. 3B, Flow cytometry binding curves of A20FMDV2, C1C18 DFBP, C2C18 DFBP, and additionally modified peptides to K562 and K562 αvβ6:mCherry cells, normalized to 400 nM A20FMDV2 binding to K562 αvβ6:mCherry cells. The curves represent a nonlinear regression of at least three independent experiments in which binding data are fitted to a Hill equation. K_{D} values were calculated by averaging the individual regression values of the independent experiments. Data points, error bars, and K_{D} values represent the mean±s.d.; n=3-8 independent experiments. K_{D} values of cyclized and modified peptides were not statistically different than that of the original peptide (P>0.05, one-way ANOVA with Dunnett's test) and each other (P>0.05, one-way ANOVA with Tukey's test). SA-AF647, streptavidin Alexa Fluor 647. C1C18 (SEQ ID NO:2); C2C18 (SEQ ID NO:5); C2C18 R_{D}TKA_{D} DFBP (SEQ ID NO:9); C2C18 CitTKA_{D} DFBP (SEQ ID NO:10); C2C18 PₕR_{D}TKA_{D} DFBP (SEQ ID NO:11); C1C18 A_{D}R_{D}TK_{D}A_{D} DFBP (SEQ ID NO:12).

FIGS. 4A through 4D. Further modified DFBP-cyclized A20FMDV2 peptides exhibit prolonged serum stability with slow internal arginine cleavage. FIGS. 4A-4D, MALDI-TOF spectra of DFBP-cyclized C2C18 R_{D}TKA_{D} (SEQ ID NO:9), C2C18 CitTKA_{D} (SEQ ID NO:10), C2C18 PₕR_{D}TKA_{D} (SEQ ID NO:11), and C1C18 A_{D}R_{D}TK_{D}A_{D} (SEQ ID NO:12) peptides incubated in normal mouse serum for 0, 4, 8, 12, and 24 h at 37° C. Molecular weights of prominent peaks are shown. Bottom: predicted amino acid sequences of degradation products based on measured molecular weights. For some molecular weights, multiple predictions are listed.

FIGS. 5A through 5D. Further modified DFBP-cyclized A20FMDV2 peptides have long and comparable serum half-lives. FIGS. 5A-5D, Stability of DFBP-cyclized C2C18 R_{D}TKA_{D} (SEQ ID NO:9), C2C18 CitTKA_{D} (SEQ ID NO:10), C2C18 PₕR_{D}TKA_{D} (SEQ ID NO:11), and C1C18 A_{D}R_{D}TK_{D}A_{D} (SEQ ID NO: 12) peptides over a 24-h incubation in normal mouse serum, as measured by LC-MS. Values are normalized to the 0 h timepoint. Curves represent a non-linear regression of one independent experiment assuming one phase exponential decay.

FIG. 6. Citrulline, hydroxyproline, and D-alanine substitutions reduced non-specific integrin binding of DFBP-cyclized A20FMDV2 peptides. Non-specific binding of DFBP-cyclized and modified peptides to A375P cells by flow cytometry, normalized to 125, 250, 500, and 1000 nM A20FMDV2 binding. Values that fall above the dotted gray line represent increased non-specific binding compared to the original A2FMDV2 peptide (SEQ ID NO:1), whereas those that fall below represent decreased non-specific binding. The gray dots represent the data from individual experiments. Columns and error bars represent the mean±s.d.; n=3 independent experiments. For statistical testing, values from independent experiments and all four concentrations were pooled together for each peptide before comparison to the original peptide (n=12, P>0.05, ***P<0.001, ****P<0.0001, paired one-way ANOVA with Dunnett's test). SA-AF647, streptavidin Alexa Fluor 647.

FIG. 7. Disulfide cyclization provides less enzymatic stability than DFBP cyclization for the C1C18 (SEQ ID NO:8) peptide. MALDI-TOF spectra of disulfide-cyclized C1C18 S-S incubated in normal mouse serum for 0, 2, 4, and 6 h at 37° C. Molecular weights of prominent peaks are shown. Bottom: predicted amino acid sequences of degradation products based on measured molecular weights.

FIG. 8. DFBP-cyclized C1C18 A_{D}R_{D}TK_{D}A_{D} is degraded into a stable 138 Da smaller product over serum incubation. Accumulation of a 138 Da smaller degradation product from the DFBP-cyclized C1C18 A_{D}R_{D}TK_{D}A_{D} (SEQ ID NO:12) peptide over a 24-h incubation in normal mouse serum, as measured by LC-MS. Values are normalized to the 0 h timepoint for the intact peptide.

FIGS. 9A through 9B. DFBP-cyclized C2C18 peptides with arginine mimetic-modified RGD motifs fail to bind αvβ6⁺ cancer cells. FIG. 9A, schematic of mimetic substitutions made to the sequence of C2C18 DFBP to replace arginine in the RGD motif. Chemical structures of arginine (black) and mimetics (gray) are shown for comparison. The resulting mimetic-substituted peptide sequences are also listed, with cysteine substitutions for DFBP cyclization shown in gray and substitutions and C-terminal modifications shown in dark gray. The RGDLXXL (SEQ ID NO:17) motif that is important for αvβ6 recognition is underlined in all sequences. FIG. 9B, flow cytometry binding curves of mimetic-substituted peptides to K562 and K562 αvβ6:mCherry cells, normalized to 400 nM A20FMDV2 binding to K562 αvβ6:mCherry cells. The curves represent a nonlinear regression of one independent experiment in which binding data are fitted to a Hill equation. SA-AF647, streptavidin Alexa Fluor 647. C2C18 _{A}FGDRTKA_{D} DFBP (SEQ ID NO:13); C2C18 CitGD DFBP (SEQ ID NO:14); and C2C18 _{G}FGDR_{D}TKA_{D} DFBP (SEQ ID NO:15).

FIGS. 10A through 10B. Modified decafluorobiphenyl (DFBP)-cyclized A20FMDV2 peptide is rapidly internalized by αvβ6⁺ pancreatic BxPC-3 cells. FIG. 10A, peptide sequence of biotinylated C2C18 PₕA_{D}R_{D}TK_{D} DFBP. Cysteine substitutions for DFBP cyclization are shown in gray and amino acid modifications are shown in dark gray. The N-terminus of the peptide is acetylated whereas biotin was selectively conjugated to the C-terminal D-lysine via its side-chain amino group. FIG. 10B, flow cytometry detection of bound DFBP-cyclized C2C18 Pₕ A_{D} R_{D}TK_{D} remaining on the surface of BxPC-3 cells over a 60-min incubation at 37° C., normalized to a 0-min no incubation control. The curve represents a nonlinear regression of one independent experiment assuming one-phase exponential decay. SA-AF647, streptavidin Alexa Fluor 647. C2C18 PₕA_{D}R_{D}TK_{D} DFBP (SEQ ID NO:16).

FIGS. 11A through 11B. Modified decafluorobiphenyl (DFBP)-cyclized A20FMDV2 peptide exhibits moderate plasma circulation half-life in mice. FIG. 11A, peptide sequence of Cy5-labeled C2C18 Pₕ A_{D} R_{D}TK_{D} DFBP. Cysteine substitutions for DFBP cyclization are shown in gray and amino acid modifications are shown in dark gray. The N-terminus of the peptide is acetylated whereas Cy5 was selectively conjugated to the C-terminal D-lysine via its side-chain amino group. FIG. 11B, Plasma half-life of Cy5-labeled C2C18 Pₕ A_{D} R_{D} TK_{D} in NOD scid mice following a 10 nmol retro-orbital (RO) injection. The curve represents a nonlinear regression assuming two-phase exponential decay. Data points and error bars represent the mean±s.d.; n=2-4 mice. Cy5, cyanine 5. C2C18 PₕA_{D}R_{D}TK_{D} DFBP (SEQ ID NO:16).

FIG. 12. Cyclization and modification of the A20FMDV2 peptide improves its serum stability and specificity without sacrificing its affinity for the integrin αvβ6. Cyclization by perfluoroarylation with decafluorobiphenyl improved serum stability. The cyclized variants combined with further modification using citrulline, hydroxyproline, and D-alanine further improved serum stability, increased specificity all without reducing binding affinity.

### DETAILED DESCRIPTION

The integrin αvβ6 is an antigen expressed at low levels in healthy tissue but upregulated during tumorigenesis, which makes it a promising target for cancer imaging and therapy. A20FMDV2 is a 20-mer peptide derived from the foot-and-mouth disease virus that exhibits nanomolar and selective affinity for αvβ6 over other integrins. Despite this selectivity, A20FMDV2 has had limited success in imaging and treating αvβ6⁺ tumors in vivo because of its poor serum stability. Here, the present disclosure describes that cyclization and modification of the A20FMDV2 peptide improves its serum stability without sacrificing its affinity and specificity for αvβ6. Using cysteine amino acid substitutions and cyclization by a perfluoroaryl compound (e.g., decafluorobiphenyl), six cyclized A20FMDV2 variants were synthesized and it was discovered that two retained binding to αvβ6 with modestly improved serum stability. Further D-amino acid substitutions and C-terminal sequence optimization outside of the cyclized region greatly prolonged peptide serum stability without reducing binding affinity. While the cyclized A20FMDV2 variants exhibited increased nonspecific integrin binding compared to the original peptide, additional modifications with the non-natural amino acids citrulline, hydroxyproline, and D-alanine were found to restore binding specificity, with some modifications leading to greater αvβ6 integrin selectivity than the original A20FMDV2 peptide. The peptide modifications detailed herein greatly improve the potential for utilization of A20FMDV2 to target αvβ6 in vivo, expanding opportunities for cancer targeting and therapy.

In accordance with the foregoing, in one aspect the disclosure provides a synthetic peptide that can comprise an amino acid sequence of X₁X₂VX₃NLRGDLQVLX₄QKVCX₅T (SEQ ID NO:18). In some embodiments, the disclosure provides a synthetic peptide that can comprise an amino acid sequence of X₁X₂VX₃NLRGDLQVLX₄QKVCX₅TX₆ (SEQ ID NO:19). In some embodiments, the disclosure provides a synthetic peptide that can comprise an amino acid sequence of X₁X₂VX₃NLRGDLQVLX₄QKVCX₅TX₆X₇ (SEQ ID NO:20). In some embodiments, at least one of X₁ and X₂ can be a cysteine.

In other embodiments, X₁ can be a cysteine. In some embodiments, at least one perfluoroaryl compound linker can connect the thiol group of the cysteine at X₁ to the thiol group of a second cysteine in the amino acid sequence as set forth in (SEQ ID NOs:18-20). In still other embodiments, the at least one perfluoroaryl compound linker connecting the thiol group of the cysteine at X₁ to the thiol group of the second cysteine can create a cyclic portion at X₁ and the second cysteine. In still other embodiments, X₂ can be a cysteine. In some embodiments, at least one perfluoroaryl compound linker can connect the thiol group of the cysteine at X₂ to the thiol group of a second cysteine in the amino acid sequence as set forth in (SEQ ID NOs: 18-20). In still other embodiments, the at least one perfluoroaryl compound linker connecting the thiol group of the cysteine at X₂ to the thiol group of the second cysteine can create a cyclic portion at X₂ and the second cysteine.

In some embodiments, one or more of X₃, X₄, X₅, X₆, and X₇ can be a non-natural amino acid. In some embodiments, one or more of X₄, X₅, X₆, and X₇ can be a non-natural amino acid. In some embodiments, one or more of X₅, X₆, and X₇ can be a non-natural amino acid. In some embodiments, one or more of X₆, and X₇ can be a non-natural amino acid. In still other embodiments, X₇ can be a non-natural amino acid. In some embodiments, one or more of X₃, X₄, X₅, and X₆ can be a non-natural amino acid. In some embodiments, one or more of X₃, X₄, and X₅ can be a non-natural amino acid. In some embodiments, one or more of X₃, and X₄ can be a non-natural amino acid. In still other embodiments, X₃ can be a non-natural amino acid. In some embodiments, X₅ can be a non-natural amino acid. In some embodiments, X₅ can be D-arginine. In other embodiments, X₅ can be citrulline.

In some embodiments, the synthetic peptide can further comprise a therapeutic agent. In some embodiments, the therapeutic agent can be bound to X₁ as set forth in SEQ ID NOs:18-20. In other embodiments, the therapeutic agent can be bound to at least one of X₁ and X₆ as set forth in SEQ ID NO:19. In still other embodiments, the therapeutic agent can be bound to at least one of X₁ and X₇ as set forth in SEQ ID NO:20. In still other embodiments, the synthetic peptide can further comprise an imaging agent. In some embodiments, the imaging agent can be bound to X₁ as set forth in SEQ ID NOs (18-20). In other embodiments, the imaging agent can be bound to at least one of X₁ and X₆ as set forth in SEQ ID NO 19. In still other embodiments, the imaging agent can be bound to at least one of X₁ and X₇ as set forth in SEQ ID NO 20.

In some embodiments, the synthetic peptide can comprise an amino acid sequence of X₁X₂VX₃NLRGDLQVLX₄QKVCX₅TX₆ as set forth in (SEQ ID NO: 19), wherein X₁ can be an asparagine; X₂ can be a cysteine; X₃ can be a hydroxyproline; X₄ can be a D-alanine; X₅ can be a D-arginine; and X₆ can be a D-lysine. In some embodiments, the synthetic peptide can further comprise a therapeutic agent bound to one or more of X₁ and X₆. In still other embodiments, the synthetic peptide can further comprise an imaging agent bound to one or more of X₁ and to X₆.

In some embodiments, the synthetic peptide can comprise an amino acid sequence of X₁X₂VX₃NLRGDLQVLX₄QKVCX₅TX₆X₇ as set forth in (SEQ ID NO:20), wherein X₁ can be an asparagine; X₂ can be a cysteine; X₃ can be a proline; X₄ can be an alanine; X₅ can be a D-arginine; X₆ can be a lysine; and X₇ can be a D-alanine. In some embodiments, the synthetic peptide can further comprise a therapeutic agent bound to one or more of X₁ and X₇. In still other embodiments, the synthetic peptide can further comprise an imaging agent bound to one or more of X₁ and to X₇.

In some embodiments, the synthetic peptide can comprise an amino acid sequence of X₁X₂VX₃NLRGDLQVLX₄QKVCX₅TX₆X₇ as set forth in (SEQ ID NO:20), wherein X₁ can be an asparagine; X₂ can be a cysteine; X₃ can be a proline; X₄ can be an alanine; X₅ can be a citrulline; X₆ can be a lysine; and X₇ can be a D-alanine. In some embodiments, the synthetic peptide can further comprise a therapeutic agent bound to one or more of X₁ and X₇. In still other embodiments, the synthetic peptide can further comprise an imaging agent bound to one or more of X₁ and to X₇.

In some embodiments, the synthetic peptide can comprise an amino acid sequence of X₁X₂VX₃NLRGDLQVLX₄QKVCX₅TX₆X₇ as set forth in (SEQ ID NO:20), wherein X₁ can be an asparagine; X₂ can be a cysteine; X₃ can be a hydroxyproline; X₄ can be an alanine; X₅ can be a D-arginine; X₆ can be a lysine; and X₇ can be a D-alanine. In some embodiments, the synthetic peptide can further comprise a therapeutic agent bound to one or more of X₁ and X₇. In still other embodiments, the synthetic peptide can further comprise an imaging agent bound to one or more of X₁ and to X₇.

In still other embodiments, the synthetic peptide can comprise an amino acid sequence of X₁X₂VX₃NLRGDLQVLX₄QKVCX₅TX₆X₇ as set forth in (SEQ ID NO:20), wherein X₁ can be cysteine; X₂ can be a D-alanine; X₃ can be a proline; X₄ can be a D-alanine; X₅ can be a D-arginine; X₆ can be a D-lysine; and X₇ is a D-alanine. In some embodiments, the synthetic peptide can further comprise a therapeutic agent bound to one or more of X₁ and X₇. In still other embodiments, the synthetic peptide can further comprise an imaging agent bound to one or more of X₁ and to X₇.

In some embodiments, the claimed synthetic peptide can be specific for binding to an integrin. In some embodiments, the integrin can be integrin αvβ6. In still other embodiments, the motif that is important for specific binding of the claimed synthetic peptide to integrin αvβ6 can be RGDLXXL, wherein X can be any amino acid, including, but not limited to alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.

In another aspect, the disclosure provides a cyclized peptide that can specifically bind to integrin αvβ6, the peptide can comprise a sequence as set forth in one of SEQ ID NOs: 2-7. In some embodiments, the cyclization can comprise a cysteine linker with at least one perfluoroaryl compound. In some embodiments, the cysteine linker with at least one perfluoroaryl compound can link a first cysteine with a second cysteine to create a cyclic peptide. In some embodiments, the first cysteine and the second cysteine can be introduced along any position of the amino acid sequence as set forth in SEQ ID NO:1. In some embodiments, the first cysteine and second cysteine are 1, 2, 3, 4, 5, 6, 7, 8, 9, 10, 11, 12, 13, 14, 15, 16, 17, 18, 19, or 20 amino acids apart (e.g., CXXXXC is four amino acids apart). In some embodiments, connecting the cysteine linker with at least one perfluoroaryl compound to the first cysteine and the second cysteine can create a cyclic peptide comprising 1, 2, 3, 4, 5, 6, 7, 8, 9, 10, 11, 12, 13, 14, 15, 16, 17, 18, 19, or 20 amino acids (e.g., CXXC would create a four amino acid cyclic peptide). In still other embodiments, the first cysteine and the second cysteine are between 10-18 amino acids apart. In some embodiments, connecting the cysteine linker with at least one perfluoroaryl compound to the first cysteine and the second cysteine can create a cyclic peptide comprising between 10-18 amino acids.

In some embodiments, the cyclized peptides as set forth in one of SEQ ID NOs: 2-7 can comprise additional modifications. In some embodiments, the modifications can include but are not limited to substitution with one or more non-natural amino acids. In some embodiments, the modification can be inside the cyclized region (e.g., the region forming the cyclic peptide). In still other embodiments, the modification can be outside the cyclized region (e.g., the unprotected tail region). In some embodiments, the modification can include substitution of a D-arginine and a D-alanine outside the cyclized region as set forth in SEQ ID NO:9. In some embodiments, the modification can include substitution of a citrulline and a D-alanine outside the cyclized region as set forth in SEQ ID NO:10. In still other embodiments, the modification can include substitution of a D-arginine and a D-alanine outside the cyclized region and substitution of a hydroxyproline within the cyclized region as set forth in SEQ ID NO:11. In some embodiments, the modification can include substitution of a D-arginine, a D-lysine, and a D-alanine outside the cyclized region and substitution of a first D-alanine and a second D-alanine within the cyclized region as set forth in SEQ ID NO: 12. In some embodiments, the modification can include substitution of a D-alanine outside the cyclized region and substitution of a 4-aminophenylalanine within the cyclized region as set forth in SEQ ID NO:13. In still other embodiments, the modification can include substitution of a citrulline within the cyclized region as set forth in SEQ ID NO:14. In some embodiments, the modification can include substitution of a D-arginine and a D-alanine outside the cyclized region and substitution of a 4-guanidinophenylalanine within the cyclized region as set forth in SEQ ID NO:15. In still other embodiments, the modification can include substitution of a D-arginine and a D-lysine outside the cyclized region and substitution of a hydroxyproline and a D-alanine within the cyclized region as set forth in SEQ ID NO:16.

In some embodiments, the cyclized peptides as set forth in one of SEQ ID NOs: 2-7 can comprise additional modifications. In some embodiments, the modification can include but are not limited to binding a therapeutic agent to one or more of a N-terminal amino acid and a C-terminal amino acid. In still other embodiments, the modification can include but are not limited to binding an imaging agent to one or more of a N-terminal amino acid and a C-terminal amino acid. In some embodiments, the modification can include but are not limited to binding a therapeutic agent to a N-terminal amino acid. In some embodiments, the modification can include but are not limited to binding a therapeutic agent to a C-terminal amino acid. In still other embodiments, the modification can include but are not limited to binding an imaging agent to a N-terminal amino acid. In still other embodiments, the modification can include but are not limited to binding an imaging agent to a C-terminal amino acid.

In another aspect, the disclosure provides a method of inhibiting growth of a cancer cell overexpressing integrin αvβ6, the method comprising administering to a patient in need thereof a therapeutically effective amount of a composition comprising a synthetic peptide that specifically binds αvβ6 and a pharmaceutically acceptable carrier.

In some embodiments, the composition comprises the synthetic peptide combined with the carrier in a first container. In some embodiments, the composition comprises the synthetic peptide in a first container and the carrier in a second container.

In some embodiments, the disclosure provides a method of inhibiting growth of a cancer cell overexpressing integrin αvβ6, the method comprising administering to a patient in need thereof a therapeutically effective amount of a composition comprising a synthetic peptide that specifically binds αvβ6, a therapeutic agent, and a pharmaceutically acceptable carrier.

In some embodiments, the composition comprises the synthetic peptide and the therapeutic agent in a first container and the carrier in a second container. In still other embodiments, the composition comprises the synthetic peptide and the therapeutic agent combined with the carrier in a first container.

In some embodiments, the method can inhibit the growth of a cancer cell associated with the overexpression of an integrin receptor. In some embodiments, the integrin receptor can be the integrin αvβ6. Non-limiting examples of cancers associated with the overexpression of the integrin αvβ6 can include solid tumor types, such as pancreatic, liver, lung, esophageal, cervical, breast, head and neck, colon, ovarian, stomach, and oral cancers.

In some embodiments, the method comprises administering to a patient in need thereof a therapeutically effective amount of a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), and a pharmaceutically acceptable carrier.

In some embodiments, the method comprises administering to a patient in need thereof a therapeutically effective amount of a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), a pharmaceutically acceptable carrier, and a therapeutic agent.

In some embodiments, the method comprises administering to a patient in need thereof a therapeutically effective amount of a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), and a pharmaceutically acceptable carrier, wherein the synthetic peptide is bound to a therapeutic agent.

In some embodiments, the therapeutic agent is any cancer therapeutic agent well-known to one of ordinary skill in the art including but not limited to cytotoxic chemotherapeutic agents.

Non-limiting examples of cytotoxic chemotherapeutic agents include but are not limited to alkylating agents including nitrogen mustards such as mechlorethamine (HN2), cyclophosphamide, ifosfamide, melphalan (L-sarcolysin) and chlorambucil; 10 ethylenimines and methylmelamines such as hexamethylmelamine, thiotepa; alkyl sulphonates such as busulfan; nitrosoureas such as carmustine (BCNU), lomustine (CCNLJ), semustine (methyl-CCN-U) and streptozoein (streptozotocin); and triazenes such as decarbazine (DTIC; dimethyltriazenoimidazolecarboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycofonnycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin Q; enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and antbracycline; substituted urea such as hydroxyurea; methyl hydrazine derivative such as procarbazine (N-methylhydrazine, MIH); and adrenocortical suppressant such as mitotane (o, p′-DDD) and aminoglutethimide; taxol and analogues/derivatives; and hormone agonists/antagonists such as flutamide and tamoxifen.

One of ordinary skill in the art will be familiar with cancer therapeutic agents and can select a therapeutic agent based on the type of cancer. In some embodiments, the therapeutic agent is bound to X₁. In some embodiments, the therapeutic agent is bound to X₆. In some embodiments, the therapeutic agent is bound to X₇. In still other embodiments, the therapeutic agent is bound to one or more of X₁, X₆, and X₇. One of ordinary skill in the art will be familiar with techniques to bind a therapeutic agent to a synthetic peptide.

In still other embodiments, the method can further comprise co-administering embodiments of the claimed composition with a second therapeutic agent, wherein the second therapeutic agent can be any cancer therapeutic agent well-known to one of ordinary skill in the art. In some embodiments, embodiments of the claimed composition are administered before the second therapeutic. In some embodiments, embodiments of the claimed composition are administered after the second therapeutic. In still other embodiments, embodiments of the claimed composition are administered concurrently with the second therapeutic.

In another aspect, the disclosure provides an in vivo method of detecting a cancer cell overexpressing integrin αvβ6, the method comprising administering to a patient a composition comprising a synthetic peptide that specifically binds αvβ6 and an imaging agent.

In some embodiments, the composition comprises the synthetic peptide and the imaging agent in a first container and the carrier in a second container. In still other embodiments, the composition comprises the synthetic peptide, the imaging agent combined with the carrier in a first container.

In some embodiments, the in vivo method can detect the growth of a cancer cell associated with the overexpression of an integrin receptor. In some embodiments, the integrin receptor can be the integrin αvβ6. Non-limiting examples of cancer cell growth that can be detected with embodiments of the claim method include solid tumor types, including pancreatic, liver, lung, esophageal, cervical, breast, head and neck, colon, ovarian, stomach, and oral cancers.

In some embodiments, the method comprises administering to a patient a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), and an imaging agent.

In some embodiments, the method comprises administering to a patient a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), and an imaging agent, wherein the synthetic peptide is bound to the imaging agent.

In some embodiments, the imaging agent can include but is not limited to radionuclides, detectable tags, fluorophores, fluorescent proteins, enzymatic proteins, and the like. One of skill in the art will be familiar with imaging agents and can select a well-known imaging agent according to the type and location of the cancer cell to be imaged. In some embodiments, the imaging agents are in vivo imaging agents. In some embodiments, the imaging agent is a dye, including but not limited to cyanine 5. In still other embodiments, the imaging agent bound to the synthetic peptide further comprises a detectable tag, such as, for example biotin, avidin, streptavidin, neutravidin, and the like.

In some embodiments, the imaging agent is bound to X₁. In some embodiments, the imaging agent is bound to X₆. In some embodiments, the imaging agent is bound to X₇. In still other embodiments, the imaging agent is bound to one or more of X₁, X₆, and X₇. One of ordinary skill in the art will be familiar with techniques to bind the imaging agent to the synthetic peptide.

In some embodiments, the method comprises administering to a patient a composition comprising a synthetic peptide comprising an amino acid sequence selected from the group of X₁X₂VX₃NLRGDLQVLX₄QKVCX₅T as set forth in (SEQ ID NO:18), X₁X₂VX₃NLRGDLQVLX₄QKVCX₅TX₆ as set forth in (SEQ ID NO:19), and X₁X₂VX₃NLRGDLQVLX₄QKVCX₅TX₆X₇ as set forth in (SEQ ID NO:20), an imaging agent, and a pharmaceutically acceptable carrier.

Methods for formulating embodiments of the claimed compositions are known to those skilled in the art (see, for example, REMINGTON'S PHARMACEUTICAL SCIENCES, 18TH ED., Mack Publishing Co., Easton, PA (1990)). The composition to be administered contains a quantity of the synthetic peptide bound with an imaging agent in a pharmaceutically effective amount for imaging a tumor, organ, or tissue or for relief of a condition being treated (e.g., synthetic peptide bound with an imaging agent), when administered in accordance with the teachings of the claimed embodiments.

Administration of the synthetic peptides of the present invention with a suitable pharmaceutical excipient as necessary can be carried out via any of the accepted modes of administration. Thus, administration can be, for example, intravenous, topical, subcutaneous, transcutaneous, transdermal, intramuscular, oral, intra-joint, parenteral, intra-arteriole, intradermal, intraventricular, intracranial, intraperitoneal, intralesional, intranasal, rectal, vaginal, or by inhalation. Moreover, where injection is to treat a tumor, administration may be directly to the tumor and/or into tissues surrounding the tumor.

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, about 0.1% to about 75%, about 0.1% to 50%, or about 0.1% to 10% by weight of the synthetic peptide bound with the imaging agent or the synthetic peptide bound with the therapeutic agent, with the remainder consisting of suitable pharmaceutical carrier and/or excipients. Appropriate excipients can be tailored to the particular composition and route of administration by methods well known in the art.

In some embodiments, the composition can be administered repeatedly, e.g., at least 2, 3, 4, 5, 6, 7, 8, or more times, or the composition may be administered by continuous infusion. In some embodiments, the composition can be formulated into a solid, semi-solid, lyophilized powder, or liquid dosage forms, such as, for example, tablets, pills, lozenges, capsules, powders, solutions, suspensions, emulsions, suppositories, retention enemas, creams, ointments, lotions, gels, aerosols, or the like, preferably in unit dosage forms suitable for simple administration of precise dosages.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Mirzaei, H. and Carrasco, M. (eds.), Modern Proteomics—Sample Preparation, Analysis and Practical Applications in Advances in Experimental Medicine and Biology, Springer International Publishing, 2016; and Comai, L, et al., (eds.), Proteomic: Methods and Protocols in Methods in Molecular Biology, Springer International Publishing, 2017, for definitions and terms of art. for definitions and terms of art. Definitions of common terms in molecular biology can be found in Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). In case of conflict, the terms in the specification will control. Additionally, more specific definitions and method for peptide engineering can be found in Ngambenjawong, C., Pineda, J. M. B., and Pun, S. H. (2016) Engineering an Affinity-Enhanced Peptide through Optimization of Cyclization Chemistry. Bioconjug. Chem. 27, 2854-2862; Sellers, D. L. Tan, J-K. Y, Pineda, J. M. B, Peeler, D. J., Porubsky, V. L, and Pun S. H. (2019) Targeting Ligands Deliver Model Drug Cargo into the Central Nervous System along Autonomic Neurons. ACS Nano 13, 10961-10971; and Kolodziej, A. F., Zhang, Z., Overoye-Chan, K., Jacques, V., and Caravan, P. (2014) Peptide Optimization and Conjugation Strategies in the Development of Molecularly Targeted Magnetic Resonance Imaging Contrast Agents BT—Therapeutic Peptides: Methods and Protocols (Nixon, A. E. ed), pp. 185-211, Humana Press, Totowa, NJ, 10.1007/978-1-62703-673-3_13.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

As used herein, the term “polypeptide,” “protein,” or “peptide” are used interchangeably and refer to a polymer in which the monomers are amino acid residues that are joined together through amide bonds. When the amino acids are alpha-amino acids, either the L-optical isomer or the D-optical isomer can be used. The term peptide as used herein encompasses any amino acid sequence and includes modified sequences such as glycoproteins. The term peptide is specifically intended to cover naturally occurring proteins, as well as those that are recombinantly or synthetically produced. As used herein, a “synthetic peptide” refers to a chemically synthesized peptide. Synthesis of peptides were performed using techniques well-known to one of ordinary skill in the art.

A “cyclic peptide” refers to a peptide in which the amino-terminus of the peptide or a side-chain on the peptide having a free amino group (e.g., lysine) is joined to the carboxyl-terminus of the peptide or a side-chain on the peptide having a free carboxyl group (e.g., aspartic acid, glutamic acid), or through thiol side-chains on the peptide by a linker (e.g., perfluoroaryl compounds).

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

The term “amino acid” includes natural amino acids and non-natural amino acids. “Stereoisomers” of an amino acid refers to mirror image isomers of the amino acids, such as L-amino acids or D-amino acids.

Natural amino acids are those encoded by the genetic code (e.g., canonical amino acids). Natural amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), and tyrosine (Tyr).

Non-natural amino acids include any amino acid that does not include one of the 20 canonical amino acids, including, for example, the D-configuration of any one of the canonical amino acids. Additionally, non-natural amino acids include without limitation, amino acid analogs, amino acid mimetics, synthetic amino acids, N-substituted glycines, and N-methyl amino acids in either the L- or D-configuration, such as citrulline, hydroxyproline, 4-aminophenylalanine, and 4-guanidinophenylalanine.

As used here, “cyclization” refers to the technique for introducing cyclic structures into peptides. Methods are well known in the art for introducing cyclic structures into the peptides of the present invention to select and provide conformational constraints to the structure that results in enhanced stability. For example, a C- or N-terminal cysteine can be added to the peptide, so that when oxidized the peptide will contain a disulfide bond, generating a cyclic peptide. Other peptide cyclizing methods include cysteine perfluoroarylation that uses perfluoroaromatic molecular linkers to staple together cysteine thiol moieties on unprotected peptides. Specifically, cysteine thiol moieties are connected with a perfluoraryl compound, such as decafluorobiphenyl (DFBP), following the methods more fully described in Alexander et. al., (Alexander M. Spokoyny, Yekui Zou, Jingjing J. Ling, Hongtao Yu, Yu-Shan Lin, and Bradley L. Pentelute Journal of the American Chemical Society 2013 135 (16), 5946-5949) and Saba et. al., (Saba Alapour, Beatriz G. de la Torre, Deresh Ramjugernath, Neil A. Koorbanally, and Fernando Albericio Bioconjugate Chemistry 2018 29 (2), 225-233), which are herein incorporated by reference. Peptides can be synthesized with a cysteine at any position along the peptide and through the use of a perfluoroaryl compound a first cysteine can be connected to a second cysteine to form a cyclic peptide. The size of the cyclic peptide is dependent on the number of amino acids between the first and the second cysteine. For example, the peptide CXXXXC has 4 amino acids between the first cysteine and the second cysteine and cyclization would result in a 4 amino acid cyclic peptide. As used here, the “cyclized region” refers to the amino acids between the first cysteine and the second cysteine. As such, the amino acids within the cyclized region refer to those amino acids between the first cysteine and the second cysteine. The amino acids outside of the cyclized region refer to those amino acids located outside of the first cysteine and the second cysteine, i.e., those amino acids located more N-terminally or C-terminally to the first cysteine and the second cysteine, respectively.

The term “therapeutically effective amount” refers to the amount of the synthetic peptide, synthetic peptide and therapeutic agent, or the composition of the present invention that is capable of achieving a therapeutic effect in a subject in need thereof. For example, a therapeutically effective amount of the synthetic peptide, synthetic peptide and therapeutic agent, or the composition of the present invention can be the amount that is capable of preventing or relieving one or more symptoms associated with a disease or disorder. Additionally, a therapeutically effective amount of the synthetic peptide, synthetic peptide and therapeutic agent, or the composition is an amount sufficient to target delivery of the synthetic peptide, synthetic peptide and therapeutic agent, or the composition to a cell expressing the integrin.

As used herein, the term “administering” includes oral administration, intravenous, intraperitoneal, intramuscular, intralesional, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. By “co-administer” it is meant that a synthetic peptide, synthetic peptide and therapeutic agent, or composition of the present invention is administered at the same time, just prior to, or just after the administration of a second drug (e.g., anticancer agent and the like).

As used herein, “detecting” refers to a change in, or occurrence of, an optical signal that is detectable either by observation or instrumentally. In some embodiments, the detectable response is radioactivity (i.e., radiation), including alpha particles, beta particles, nucleons, electrons, positrons, neutrinos, and gamma rays emitted by a radioactive substance such as a radionuclide. In other embodiments, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ).

As used herein “bound” or “synthetic peptide bound to a therapeutic/imaging agent” refers to any means of connecting the agent to the synthetic peptide, which can be reversible or irreversible. One of skill in the art will be familiar with how to bind an agent to a synthetic peptide and can select the method most appropriate for the synthetic peptide, agent to be used, or selected use of the synthetic peptide bound with an agent.

As used herein, the term “unit dosage form” refers to physically discrete units suitable as unitary dosages for human subjects and other mammals (e.g., dogs), each unit containing a predetermined quantity of active material calculated to produce the desired onset, tolerability, and/or therapeutic effects, in association with a suitable pharmaceutical excipient. In addition, more concentrated compositions may be prepared, from which the more dilute unit dosage compositions may then be produced.

Reference to sequence identity addresses the degree of similarity of two polymeric sequences, such as protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

This Example describes the design of cyclized A20FMDV2 variants with selective amino acid modifications and their characterization in vitro. This Example demonstrates that these peptide variants have prolonged stability in serum and retain their binding affinity for αvβ6⁺ cells. Importantly, some of these optimized peptides demonstrate improved αvβ6 specificity over the original A20FMDV2 peptide, further increasing the benefit for future in vivo application.

### Results

### Synthesis of DFBP-Cyclized A20FMDV2 Variants and Binding Evaluation

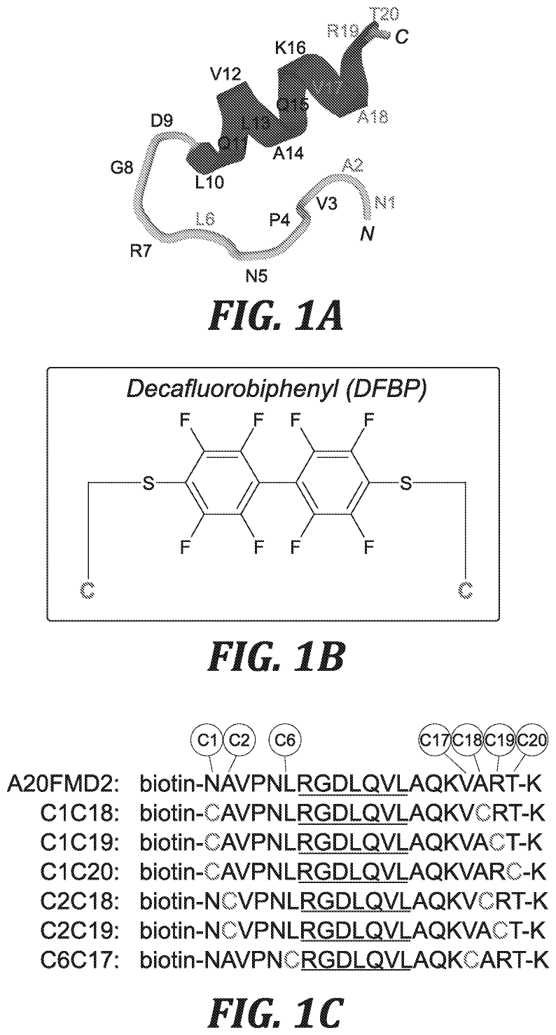

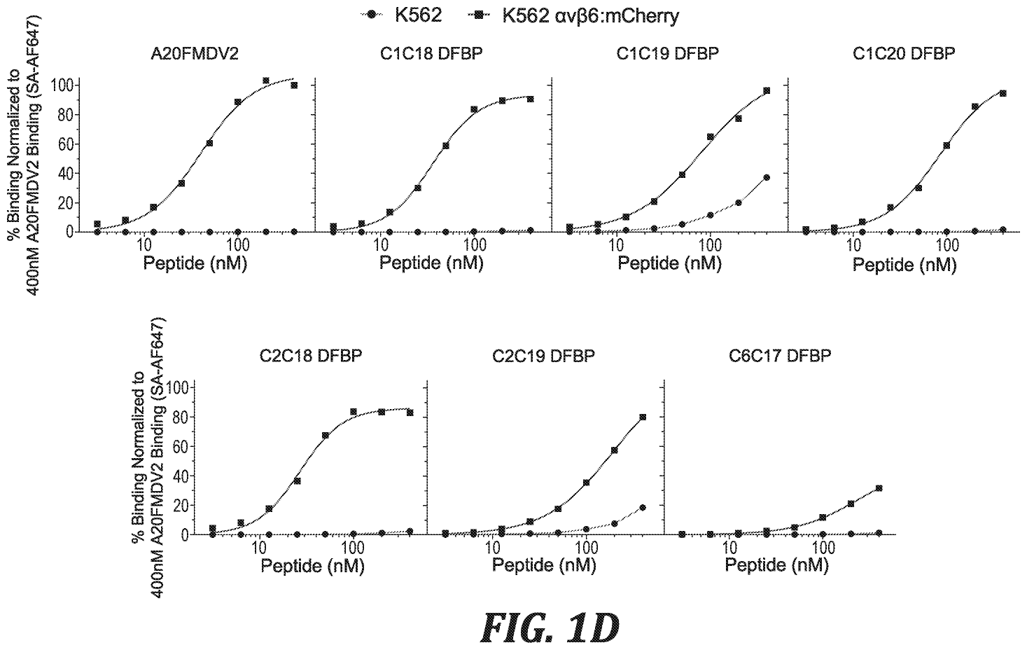

A20FMDV2 has a hairpin loop structure with the RGD motif at the tip of hairpin turn followed by a 3₁₀ helix (FIG. 1A) and previous reports have demonstrated that the extended RGDLXXL (SEQ ID NO: 17) motif is most critical for αvβ6 binding whereas amino acids at the N-terminus and C-terminus of the peptide are not as critical for binding. Accordingly, it was postulated that chemistries involving the N- and C-terminal amino-acid positions of the peptide could increase serum stability without negatively affecting peptide binding to αvβ6.

Cyclization is a well-established technique for stabilizing peptides and improving their pharmacokinetic profiles, and cysteine perfluoroarylation is a facile cyclization approach that uses perfluoroaromatic molecular linkers to staple together cysteine thiol moieties on unprotected peptides (Spokoyny, A. M., Zou, Y., Ling, J. J., Yu, H., Lin, Y.-S., and Pentelute, B. L. (2013) A Perfluoroaryl-Cysteine SNAr Chemistry Approach to Unprotected Peptide Stapling. J. Am. Chem. Soc. 135, 5946-5949). The inventors have previously used this technique for peptide cyclization with a decafluorobiphenyl (DFBP) linker (FIG. 1B) and demonstrated increased serum stability and affinity of DFBP-cyclized peptides compared to counterparts with disulfide, amide, or triazole cyclization (Ngambenjawong, C., Pineda, J. M. B., and Pun, S. H. (2016) Engineering an Affinity-Enhanced Peptide through Optimization of Cyclization Chemistry. Bioconjug. Chem. 27, 2854-2862; Sellers, D. L., Tan, J.-K. Y., Pineda, J. M. B., Peeler, D. J., Porubsky, V. L., Olden, B. R., Salipante, S. J., and Pun, S. H. (2019) Targeting Ligands Deliver Model Drug Cargo into the Central Nervous System along Autonomic Neurons. ACS Nano. 13, 10961-10971). As such, six unique A20FMDV2 peptide sequences with cysteine substitutions primarily at N- and C-terminal amino-acid positions were synthesized for cyclization by DFBP to stabilize and close the hairpin peptide structure (FIG. 1C). Biotin was conjugated on the N-terminus of all peptides for labeling with streptavidin-AF647 to assess cell binding by flow cytometry. Peptides were also synthesized with a C-terminal lysine to mimic a prospective methyltrityl-lysine that could be added for selective modification or synthesis of branched peptides at the lysine side-chain.

The binding of cyclized A20FMDV2 sequences to αvβ6 were evaluated with the matched erythroleukemia K562 and K562 αvβ6:mCherry cell lines. Both cell lines endogenously express the α5β1 integrin, but only the K562 αvβ6:mCherry cells express the αvβ6 integrin. Of the DFBP-cyclized sequences, binding of the C1C18 DFBP (N1C; A18C), C1C20 DFBP (N1C; T20C), and C2C18 DFBP (A2C; A18C) peptides was observed to K562 αvβ6:mCherry cells with high affinity and specificity comparable to the original A20FMDV2 peptide (FIG. 1D). Interestingly, peptide sequences C1C19 DFBP (N1C; R19C) and C2C19 DFBP (A2C; R19C) exhibited high binding to K562 αvβ6:mCherry cells but also poor specificity. Both peptides significantly bound to parental K562 cells at high concentrations, suggesting that the R19 amino acid in A20FMDV2 is important for αvβ6 specificity. Similarly, the peptide cyclized via cysteine substitutions proximal to the RGDLXXL (SEQ ID NO:17) motif, C6C17 DFBP (L6C; V17C), displayed minimal binding to K562 αvβ6:mCherry cells. It is known that the V12 and V17 amino acids are important for the structure of the post-RGD helix in A20FMDV2, so the cysteine substitutions in the C6C17 DFBP peptide and their cyclization likely impaired the 3₁₀ helix structure. Given that the DFBP-cyclized C1C18, C1C20, and C2C18 peptides retained the favorable binding properties of the original A20FMDV2 peptide, these variants were further characterized for serum stability.

### MALDI-ToF MS Serum Stability of DFBP-Cyclized A20FMDV2 Variants

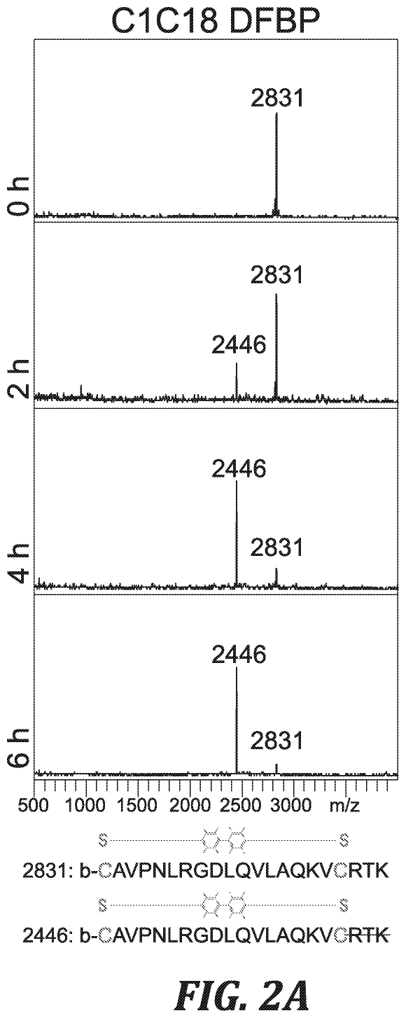

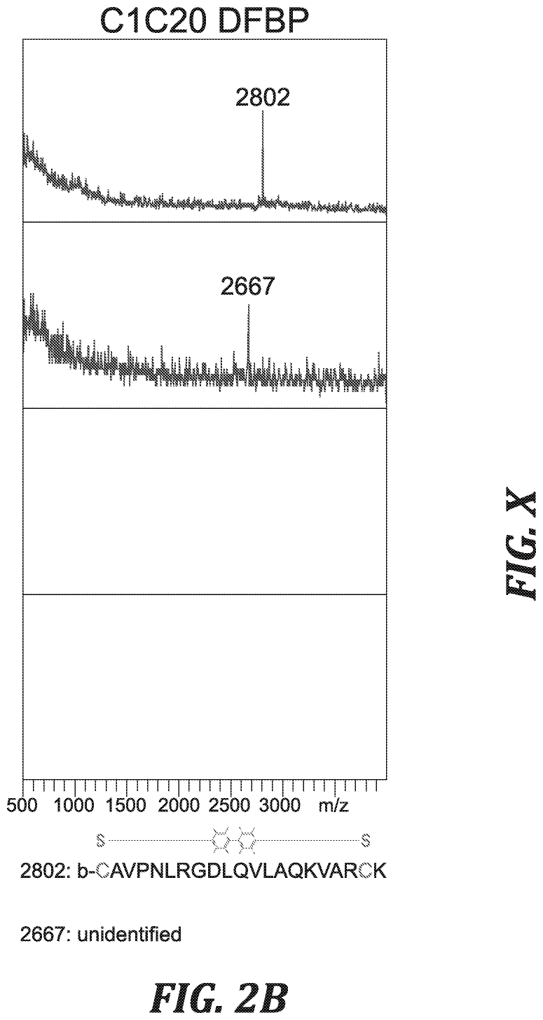

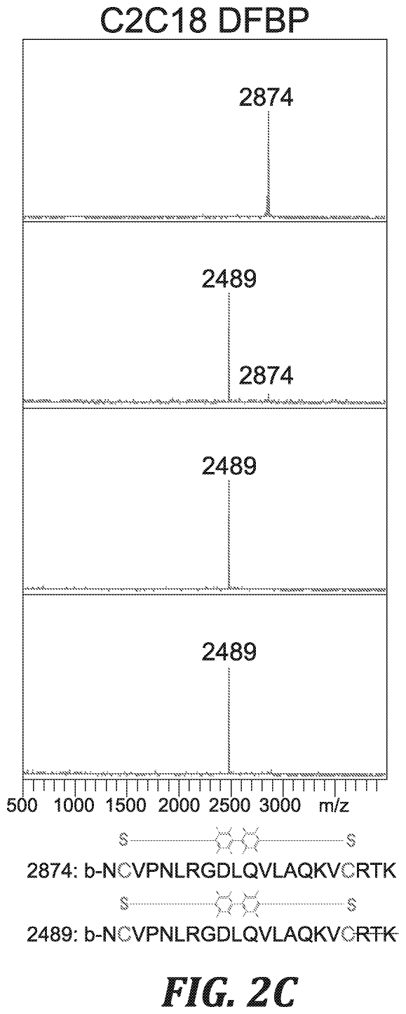

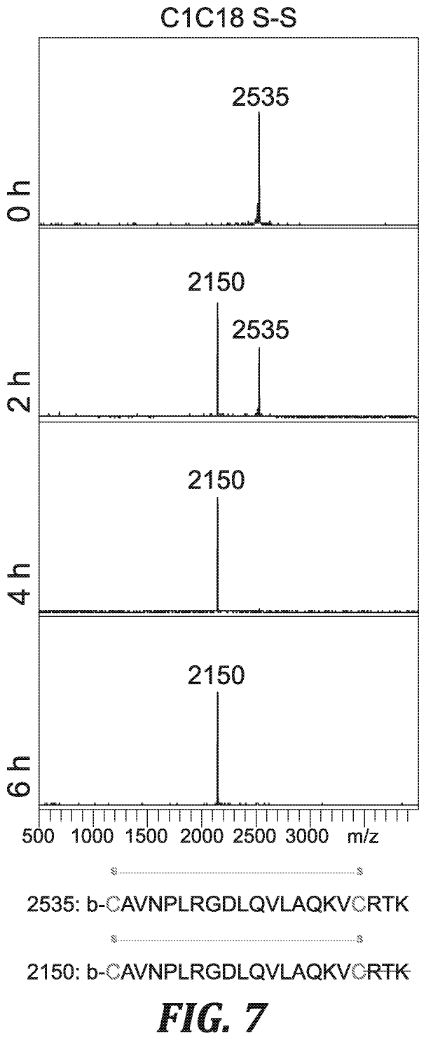

As the original A20FMDV2 peptide is degraded by over 50% in normal mouse serum within a 4 h incubation at 37° C., it was hypothesized that the DFBP-cyclized variants would have prolonged serum stability due to added structural stability from their cyclization. To investigate this, the DFBP-cyclized C1C18, C1C20, and C2C18 peptides were incubated in normal mouse serum a 37° C. for up to 6 h and measured for the presence of intact peptide and any degradation products at different time points by MALDI-ToF MS (FIG. 2A-C). As shown, the partially cyclized C1C18 DFBP and C2C18 DFBP peptides formed degradation products that are 385 Da smaller after incubation in serum for 2 h, corresponding C-terminal cleavage of the arginine-threonine-lysine (RTK) group outside of the DFBP-cyclized region (FIGS. 2A and 2C). C1C18 DFBP had prolonged intact peptide presence compared to C2C18 DFBP (6 h versus 2 h), suggesting that the C1C18 cyclization scheme better protects the exocyclic C-terminal RTK group. Importantly, the 385 Da smaller degradation products for both peptides persisted up to the last timepoint sampled and no further degradation peaks were observed, demonstrating good protection of amino acids within their cyclized regions. Additionally, a disulfide-cyclized C1C18 peptide (C1C18 S-S) was also assayed and the results demonstrate faster degradation to the 385 Da smaller product compared to C1C18 DFBP (FIG. 7), emphasizing the importance of the DFBP molecular linker.

Surprisingly, the fully cyclized C1C20 DFBP peptide degraded within 2 h in serum; peptide or degradation products were not detected at the 4 and 6 h timepoints (FIG. 2B). Detection of a low-intensity 135 smaller Da degradation product occurred at the 2 h timepoint but it was near background and the amino acid sequence was not predictable. The molecular weight difference is near what would be expected from an internal arginine deletion (138 Da smaller), so Arg7 or Arg19 may have been metabolically cleaved from the sequence. This data suggests that the complete cyclization of a peptide from N- to C-terminus is not always beneficial for stability, and that peptide structure should rationally guide cyclization positioning. Since peptides C1C18 DFBP and C2C18 DFBP were stable outside of the exocyclic C-terminal RTK groups, amino acid modification will be used to further optimize these two peptides.

### Modification of DFBP-Cyclized C1C18 and C2C18 Variants and Binding Characterization

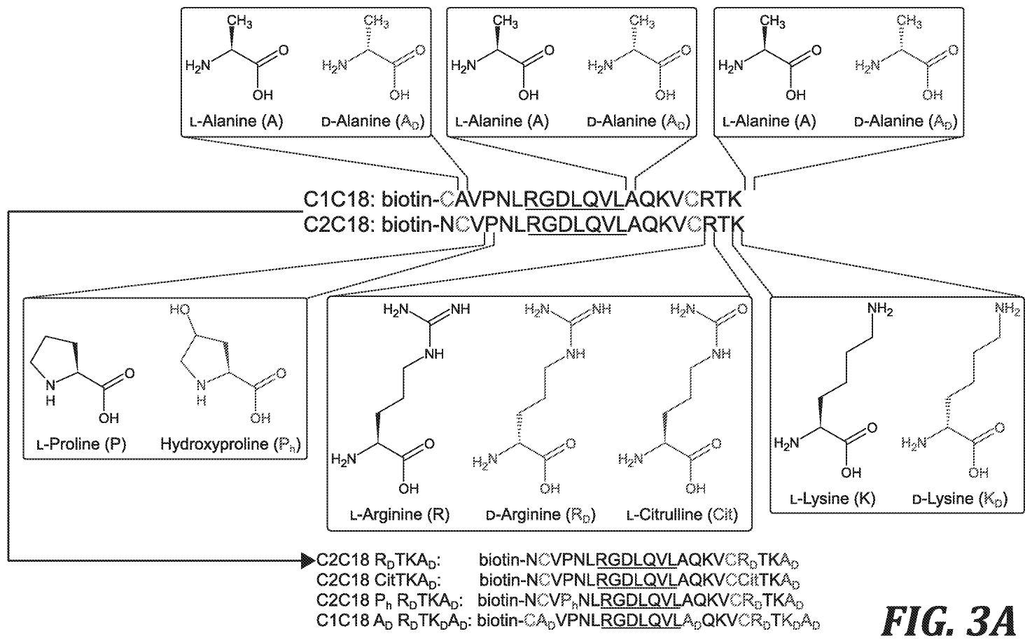

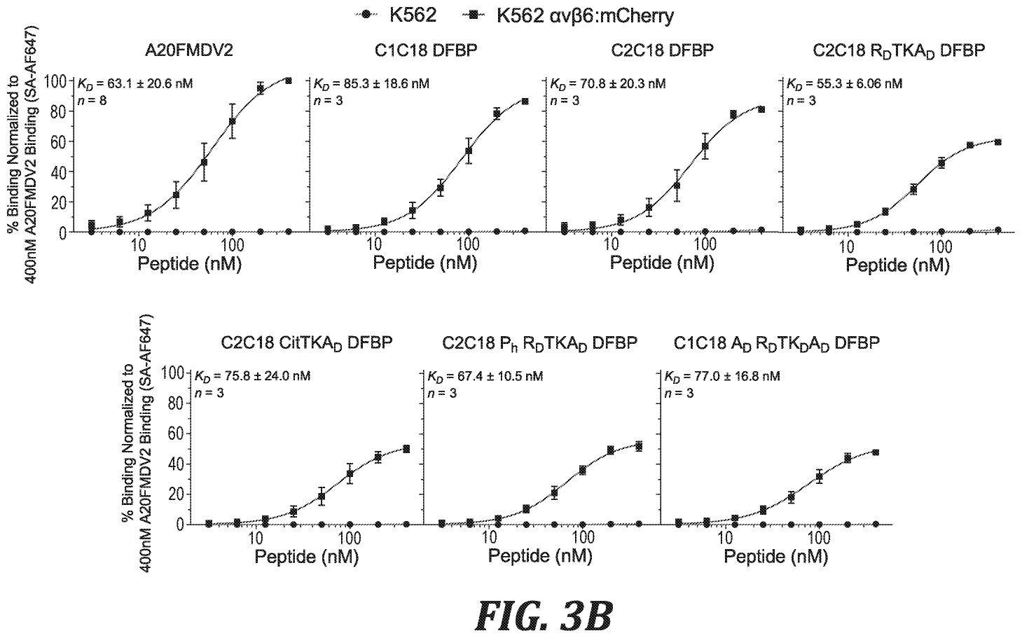

To further stabilize the DFBP-cyclized C1C18 and C2C18 peptides against proteolytic degradation, non-proteinogenic D-amino acids and naturally occurring analogs were used for additional amino acid modification (FIG. 3A). As other groups have shown increased serum stability of peptides flanked with D-amino acids (Tugyi, R., Uray, K., Iván, D., Fellinger, E., Perkins, A., and Hudecz, F. (2005) Partial D-amino acid substitution: Improved enzymatic stability and preserved Ab recognition of a MUC2 epitope peptide. Proc. Natl. Acad. Sci. U.S.A 102, 413-418), the C-terminus of our peptides was capped with D-alanine (A_{D}) in an effort to reduce degradation of the exocyclic C-terminal RTK group. Moreover, as the exocyclic C-terminal arginine is readily cleaved by many endopeptidases, the C-terminal arginine was substituted with D-arginine (R_{D}) or the nonprotein precursor 1-citrulline (Cit) as a way to further limit degradation. These peptide modifications resulted in the synthesis of DFBP-cyclized C2C18 R_{D}TKA_{D} (R19R_{D}; +22A_{D}) and C2C18 CitTKA_{D} (R19Cit; +22A_{D}) for testing. Attempts to enhance the binding kinetics of the cyclized peptides were made by substituting the N-terminal proline within the cyclized region with hydroxyproline (Pₕ), since this modification has previously demonstrated affinity improvements for other peptides. This led to the synthesis of DFBP-cyclized C2C18 Pₕ R_{D}TKA_{D} (P4Pₕ; R19R_{D}; +22A_{D}) for comparison against C2C18 R_{D}TKA_{D} DFBP which lacks the hydroxyproline group. Lastly, as the alanines within the cyclized region and the exocyclic C-terminal lysine likely do not contribute to peptide binding, these amino acids were substituted with D-alanine and D-lysine (K_{D}), respectively, for additional proteolytic stability. Accordingly, DFBP-cyclized C1C18 A_{D} R_{D}TK_{D}A_{D} (A2A_{D}; A14A_{D}; R19R_{D}; K21K_{D}; +22A_{D}) was created to reflect these changes. In total, four modified versions of either DFBP-cyclized C1C18 or C2C18 were synthesized for subsequent binding characterization.

As detailed previously, the K562 cell-binding of these newly modified variants was compared against the original A20FMDV2 peptide and the parental DFBP-cyclized C1C18 and C2C18 peptides. As shown, all DFBP-cyclized modified peptides retain selective binding to K562 αvβ6:mCherry cells with negligible background binding to K562 parental cells (FIG. 3B). Notably, the apparent binding affinities of these modified peptides (C2C18 R_{D}TKA_{D} DFBP: 55.3±6.06 nM; C2C18 CitTKA_{D} DFBP: 75.8±24.0 nM; C2C18 Pₕ R_{D}TKA_{D} DFBP: 67.4±10.5 nM; C1C18 A_{D} R_{D}TK_{D}A_{D} DFBP: 77.0±16.8 nM), as well as those of the parental DFBP-cyclized C1C18 and C2C18 peptides (85.3±18.6 nM and 70.8±20.3 nM, respectively), did not statistically differ from the affinity of the original A20FMDV2 peptide (63.1±20.6 nM). Less maximal fluorescence signal was observed for the newly modified peptides compared to A20FMDV2, C1C18 DFBP, and C2C18 DFBP. Given their similar affinities, these differences are attributed to reduced fluorescence quantum yield or biotin accessibility of the streptavidin Alexa Fluor 647 stain. Also of importance, the binding affinities observed here differ from the single digit nanomolar values reported for A20FMDV2 previously (Slack, R. J., Hafeji, M., Rogers, R., Ludbrook, S. B., Marshall, J. F., Flint, D. J., Pyne, S., and Denyer, J. C. (2016) Pharmacological Characterization of the αvβ6 Integrin Binding and Internalization Kinetics of the Foot-and-Mouth Disease Virus Derived Peptide A20FMDV2. Pharmacology. 97, 114-125). These discrepancies likely stem from differences in the binding model (cells versus purified recombinant proteins) and conditions (buffer, temperature, time) used for peptide characterization.

There were no obvious differences when comparing the larger effects of the cyclization schemes and modifications on binding affinity. DFBP-cyclized C1C18 and C2C18 peptides exhibited equivalent apparent binding affinity for K562 αvβ6:mCherry cells, and further stabilizing the C-terminus with D-arginine, citrulline, D-lysine, and/or D-alanine did not impact affinity. Interestingly, substituting proline for hydroxyproline and alanine for D-alanine within the cyclized region did not impact apparent binding affinity, suggesting that these amino acids are not critical for high-affinity αvβ6 binding. The effects of these cyclization schemes and modifications on peptide specificity for the αvβ6 integrin will be evaluated in a later section. Considering that all the newly modified DFBP-cyclized peptides retained their binding affinity for αvβ6⁺ cells, these peptides were used for qualitative and quantitative assessment of serum stability.

### MALDI-ToF MS Serum Stability of Additionally Modified C1C18 and C2C18 DFBP

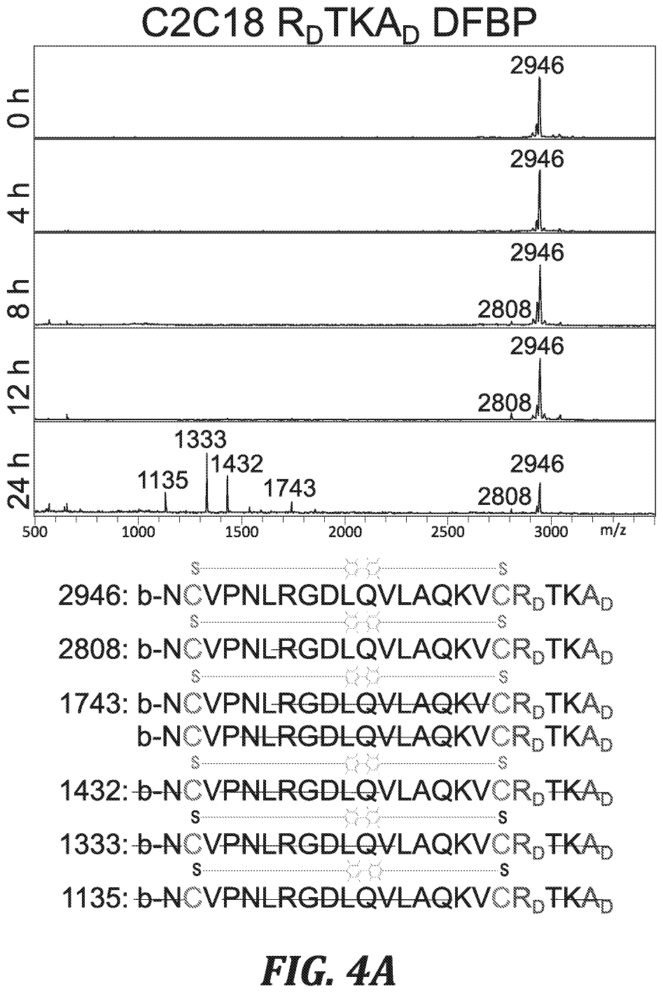

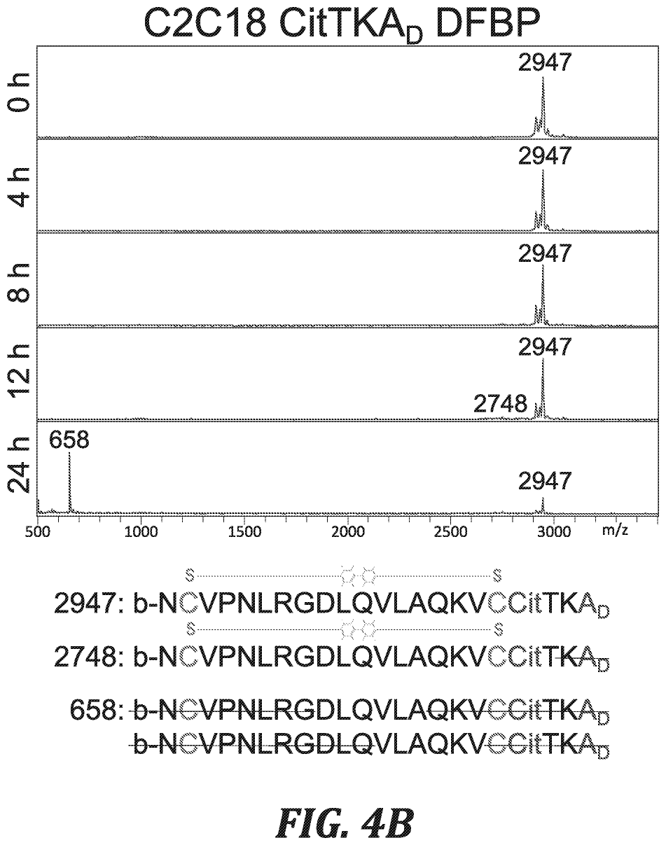

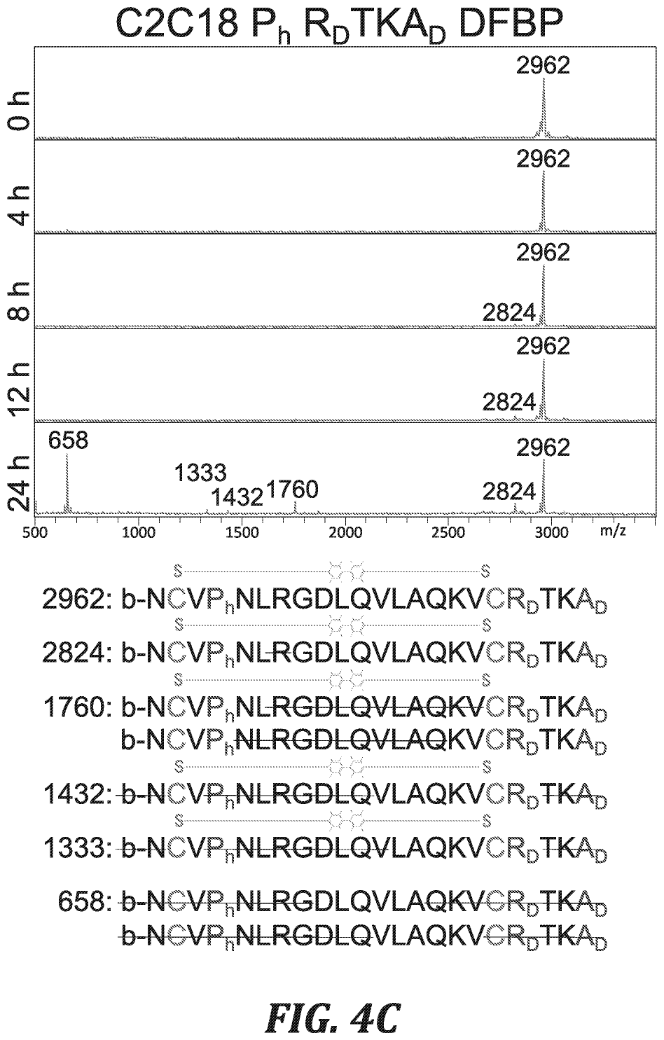

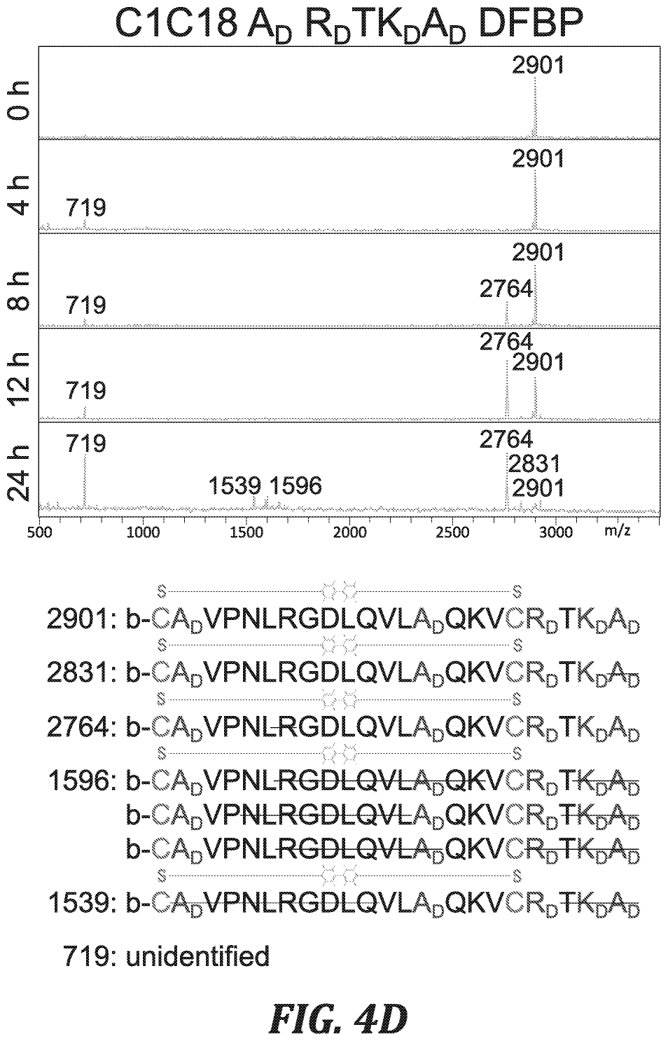

The serum stability of the newly modified peptides was assessed by MALDI-ToF MS. Fully intact peptide was still present after 24 h for each of the newly modified peptides (FIG. 4A-D), a large improvement compared to the original DFBP-cyclized C1C18 and C2C18 peptides that were degraded completely within 6 h. Nonetheless, degradation products were still detected at later time points and the relative intact peptide signal decreased over time, an indication of slow degradation of each of the peptides. Interestingly, Arg7 of the RGD motif appeared to be a primary target/site for proteolytic cleavage, as a 138 Da smaller degradation product was seen after 8 h for C2C18 R_{D}TKA_{D} DFBP, C2C18 Pₕ R_{D}TKA_{D} DFBP, and C1C18 A_{D} R_{D}TK_{D}A_{D} DFBP (FIGS. 4A and 4C-D). Many degradation products at 24 h show peptides missing even larger fragments between the cysteine-linkages, suggesting that the initial Arg7 cleave accelerated endopeptidase activity. Additionally, the relative signal of the 138 Da smaller degradation product increased over time for C1C18 A_{D} R_{D}TK_{D}A_{D} DFBP (FIG. 4D), which may signify that the C1C18 cyclization scheme better prevents further internal degradation than the C2C18 configuration after initial Arg7 cleavage. Outside of the cyclized region, N-terminal cleavage of the biotinylated Asn1 was observed for some of the modified C2C18 peptides (FIGS. 4A and 4C) as well as C-terminal cleavage at Thr20 for all the modified peptides (FIG. 4A-D). While further peptide design addressing these observed cleavage products warrants future research, the presence of intact cyclized peptides after 24 h in serum demonstrates markedly improved serum stability.

### LC-MS Serum Stability of Additionally Modified C1C18 and C2C18 DFBP

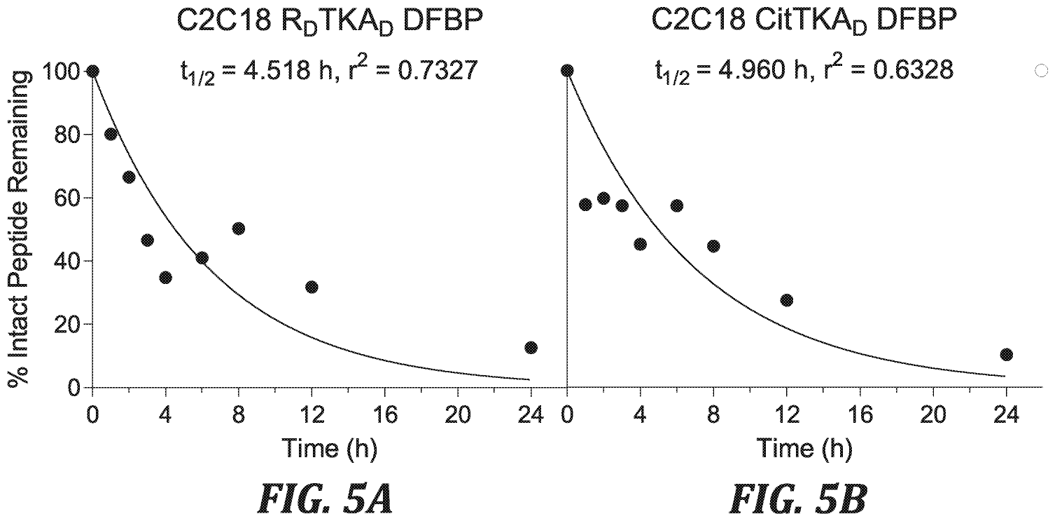

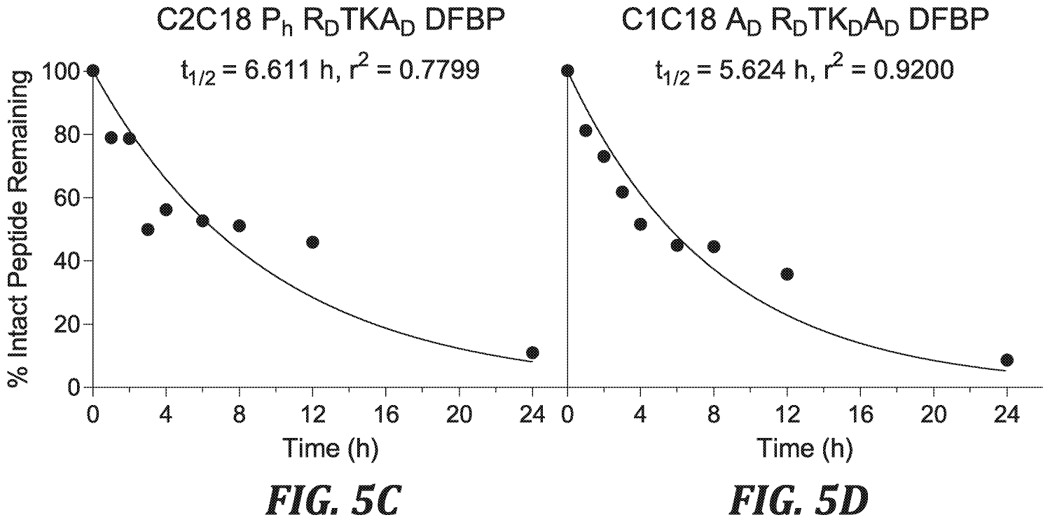

Since MALDI-ToF MS results are qualitative, the rate of degradation in normal mouse serum of the modified DFBP-cyclized C2C18 R_{D}TKA_{D}, C2C18 CitTKA_{D}, C2C18 Pₕ R_{D}TKA_{D}, and C1C18 A_{D} R_{D}TK_{D}A_{D} peptides was quantified by LC-MS. Peptides were prepared, treated in serum, and extracted as they were for the MALDI-ToF MS studies before submission for LC-MS analysis. While some of the data points are erratic due to variability in peptide extraction after acetonitrile precipitation of serum proteins, the LC-MS results were reasonably fit to a one phase exponential decay model (FIG. 5). Consistent with the MALDI results, the DFBP-cyclized and additionally modified peptides were found to degrade slowly in serum, with serum half-lives of intact peptides stretching between 4.5-6.6 h. After 24 h, the amount of remaining fully intact peptide in serum for DFBP-cyclized C2C18 R_{D}TKA_{D}, C2C18 CitTKA_{D}, C2C18 Pₕ R_{D}TKA_{D}, and C1C18 A_{D} R_{D}TK_{D}A_{D} ranged tightly at 12.6%, 10.4%, 10.9%, and 8.6%, respectively, demonstrating that they have similar basal stabilities. These results broadly suggest that citrulline and D-arginine substitutions at the peptide C-terminus (R19Cit and R19R_{D}, respectively) confer comparable proteolytic stabilities, whereas substitutions with hydroxyproline and D-alanine in the cyclized region (P4Pₕ, A2A_{D}, and A14A_{D}, respectively) have negligible impact on intact peptide serum stability.

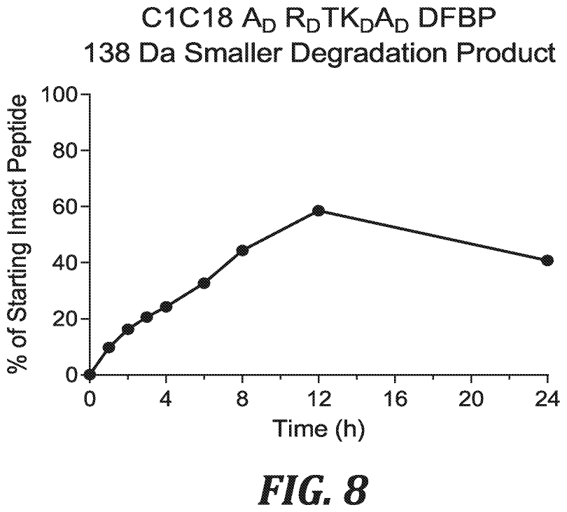

For C1C18 A_{D} R_{D}TK_{D}A_{D} DFBP specifically, LC-MS also revealed significant accumulation of the 138 Da smaller degradation product that was identified previously by MALDI-TOF (FIG. 8). After 12 h in serum, the amount of the 138 Da smaller degradation product peaked, representing nearly 60% of the starting (t=0 h) fully intact peptide; even after 24 h in serum, the presence of the degradation product was still significant, representing over 40% of the starting fully intact peptide. These data indicate that internal cleavage of Arg7 from the RGD motif is a primary degradation pathway for these modified peptides and that the resulting product remains relatively stable for the C1C18 A_{D} R_{D}TK_{D}A_{D} DFBP formulation despite the cleavage. Thus, even more stable cyclized peptides could be potentially made by further modifying the Arg7 in the RGD motif.

### Assessment of Arginine Mimetics for Modification of the RGD Motif

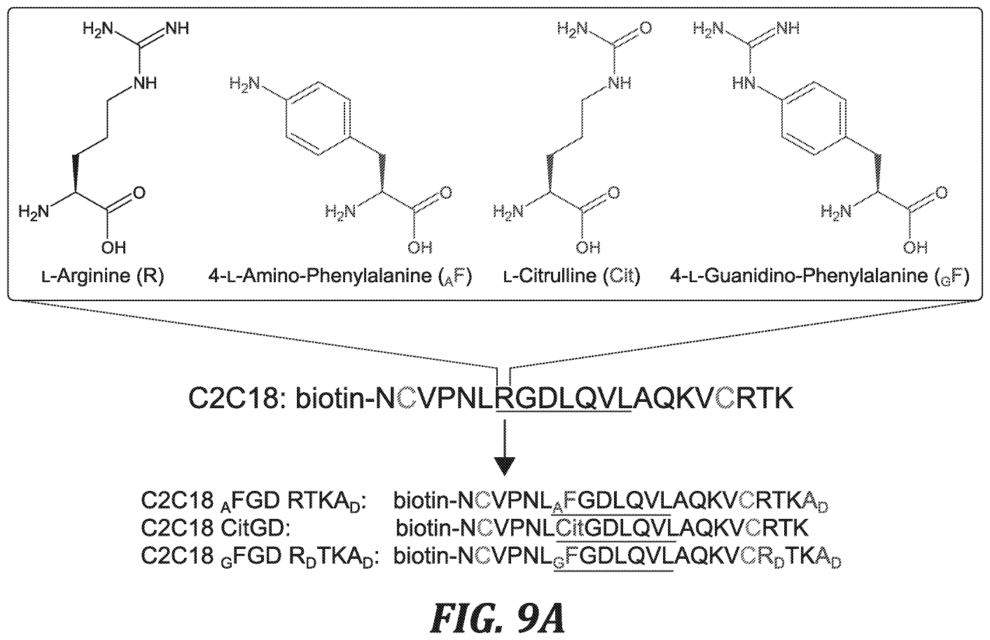

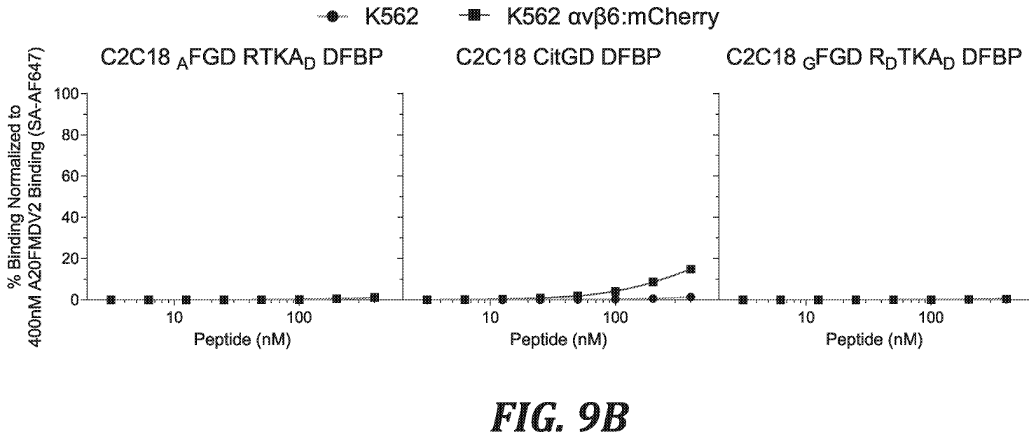

Given the previous observations, the inventors were interested in substituting arginine within the RGD motif with non-proteinogenic mimetics to prolong the already enhanced stability of our modified DFBP-cyclized A20FMDV2 variants. Phenylalanine analogs have previously demonstrated to be good mimetics for arginine replacement in other peptides (Weigel, L. F., Nitsche, C., Graf, D., Bartenschlager, R., and Klein, C. D. (2015) Phenylalanine and Phenylglycine Analogues as Arginine Mimetics in Dengue Protease Inhibitors. J. Med. Chem. 58, 7719-7733). Three versions of DFBP-cyclized C2C18 containing either 4-amino-1-phenylalanine (_{A}F), citrulline, or 4-guanidino-1-phenylalanine (_{G}F) as substitutions for Arg7 within the RGD motif were synthesized (FIG. 9A). These peptides were respectively named C2C18 _{A}FGD RTKA_{D} DFBP, C2C18 CitGD DFBP, and C2C18 _{G}FGD R_{D}TKA_{D} DFBP. The three peptides have varying degrees of C-terminal modifications, as some of these peptides were synthesized before discovering the benefit of having D-amino acids at the C-terminus. These differences were assumed not to affect the evaluation of binding potential since earlier results showed that these modifications do not influence peptide affinity for αvβ6.

C2C18 _{A}FGD RTKA_{D} DFBP and C2C18 _{G}FGD R_{D}TKA_{D} DFBP completely lost the ability to bind K562 αvβ6:mCherry cells, whereas C2C18 CitGD DFBP retained some selective binding to these cells at high concentrations, albeit low and non-saturating (FIG. 9B). Owing to the highly conserved nature of the RGD motif in integrin binding, these negative results are not surprising. Other groups have also reported drastic loss of binding for an αvβ3-specific peptide when replacing arginine in the RGD motif with closely resembling homologues (Wang, Y., Xiao, W., Zhang, Y., Meza, L., Tseng, H., Takada, Y., Ames, J. B., and Lam, K. S. (2016) Optimization of RGD-Containing Cyclic Peptides against αvβ3 Integrin. Mol. Cancer Ther. 15, 232-240). Nonetheless, the small binding observed with a citrulline substitution is promising, warranting future investigation with a more expansive panel of mimetics and analogs.

### Non-Specific Binding of DFBP-Cyclized and Modified A20FMDV2 Peptides to A375P Cells

As there are eight known integrins that recognize the RGD motif (αvβ1, αvβ3, αvβ5, αvβ6, αvβ8, α5β1, α8β1, and αIIbβ3), it is critical that RGD-containing peptides developed for diagnostic and therapeutic applications are highly specific for their targeted integrin with minimal off-target binding. The matched K562 cell model only expresses the α5β1 integrin, and thus does not rigorously test the non-specific binding of the engineered A20FMDV2 peptides to other RGD-recognizing integrins closely related to αvβ6. Accordingly, the non-specific integrin binding of our serum-stabilized peptides was compared against the original A20FMDV2 peptide and the parental DFBP-cyclized C1C18 and C2C18 peptides to melanoma A375P cells, which expresses the αvβ3, αvβ5, αvβ8, and α5β1 integrins but not the 6 integrin. For this comparison, A375P cells were used as a proxy to distinguish how the peptide cyclizations and modifications affect promiscuous integrin binding. To this end, the binding of A20FMDV2 and DFBP-cyclized C1C18, C2C18, C2C18 R_{D}TKA_{D}, C2C18 CitTKA_{D}, C2C18 Pₕ R_{D}TKA_{D}, and C1C18 A_{D} R_{D}TK_{D}A_{D} to A375P cells were evaluated at four high peptide concentrations (125, 250, 500, and 1000 nM) by flow cytometry.

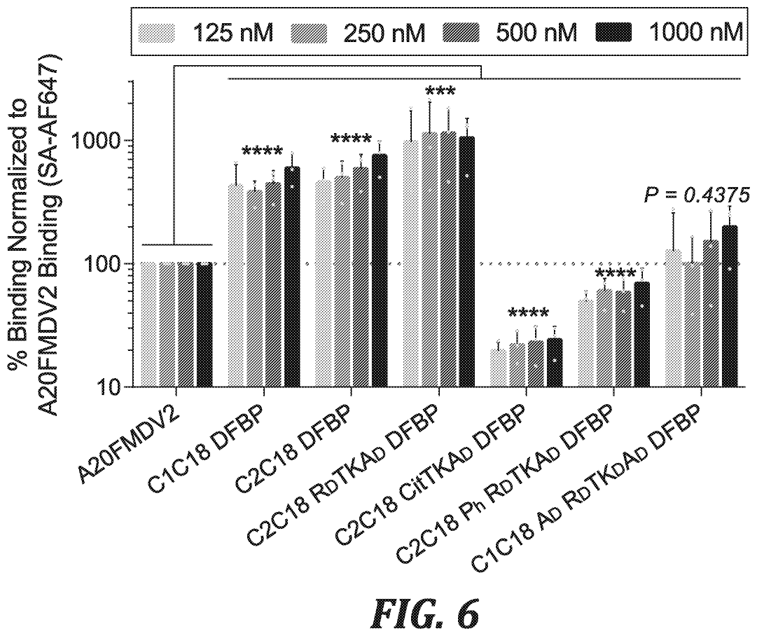

As disclosed in this Example, it is demonstrated that DFBP-cyclized C1C18 and C2C18 exhibit high non-specific binding for A375P cells, with on average 462±158% and 575±196% binding, respectively, relative to the original A20FMDV2 peptide across all four concentrations (FIG. 6). These results are consistent with findings made by Wagstaff et. al. (Wagstaff, J. L., Rowe, M. L., Hsieh, S.-J., DiCara, D., Marshall, J. F., Williamson, R. A., and Howard, M. J. (2012) NMR relaxation and structural elucidation of peptides in the presence and absence of trifluoroethanol illuminates the critical molecular nature of integrin αvβ6 ligand specificity. RSC Adv. 2, 11019-11028), which showed that disulfide cyclization of the A20FMDV2 peptide at certain positions altered the backbone dynamics of the peptide, driving loss of αvβ6 specificity. The only exception that Wagstaff et. al., found not to affect A20FMDV2 specificity was a complete N-to-C terminal disulfide cyclization, however, as disclosed in this Example, this cyclization scheme likely underwent rapid degradation in serum (FIG. 2B, C1C20 DFBP). Interestingly, even higher non-specific binding was shown for C2C18 R_{D}TKA_{D} DFBP (1072±612% on average relative to A20FMV2), suggesting that C-terminal substitution with D-arginine (R19R_{D}) has structural effects that aggravate promiscuous integrin binding.

Most notably, DFBP-cyclized C2C18 CitTKA_{D}, C2C18 Pₕ R_{D}TKA_{D}, and C1C18 A_{D} R_{D}TK_{D}A_{D} peptides did not display high non-specific binding, showing on average 22±6%, 60±16%, 145±96% binding relative to the original A20FMDV2 peptide, respectively, across all four concentrations. While some of these effects may be attributed to the reduced binding fluorescence observed previously with these peptides (˜50%, FIG. 3B), it does not completely account for their low non-specific binding, especially for C2C18 CitTKA_{D} DFBP. Furthermore, the C2C18 R_{D}TKA_{D} DFBP peptide has high non-specific binding despite also exhibiting reduced binding fluorescence (˜60%, FIG. 3B), so these two observations do not always track with each other. This data thus suggests that the electrostatic effects of the citrulline substitution (Cit19R_{D}), the hydrogen bonding or stereoelectronic effects of the hydroxyproline substitution (P4Pₕ), and the structural effects of the D-alanine substitutions (A2A_{D} and A14A_{D}) offset the high non-specific integrin binding observed for the DFBP-cyclized C1C18, C2C18, and C2C18 R_{D}TKA_{D} peptides, with some (Cit19R_{D}) instilling even improved αvβ6 integrin specificity compared to the original A20FMDV2 peptide. Combined with their greatly enhanced serum stability, the DFBP-cyclized C2C18 CitTKA_{D}, C2C18 Pₕ R_{D}TKA_{D}, and C1C18 A_{D} R_{D}TK_{D}A_{D} peptides demonstrate the potential to increase αvβ6 targeting for high-fidelity in vivo diagnostic and therapeutic applications.

### Discussion

The use of peptides for cancer targeting is limited by their rapid degradation from proteases in vivo. High and repeated doses of peptides or PEGylation are thus often required to achieve sufficient delivery to the tumor site, which can negatively impact the sensitivity and safety of medical applications that use these ligands. The virus-derived A20FMDV2 peptide, despite its high affinity and specificity for the tumor-associated integrin αvβ6, is one such peptide that suffers from poor proteolytic stability, hindering its translation into the clinic for cancer imaging and therapy. Modification of A20FMDV2 to bolster its resistance against proteases is difficult due to the highly adapted and conserved structure of the peptide; its 20-amino acid sequence contains a short β sheet, followed by the tripeptide RGD motif, and then lastly a longer 3₁₀ helix that together forms a loop structure. The amino acids in the β sheet and 3₁₀ helix that flank the RGD motif strongly influence the affinity and specificity of A20FMDV2 for αvβ6, and thus modifying these amino acids runs the risk of compromising either or both of these attributes that are equally critical for the peptide's successful translation.

As disclosed in this Example, a panel of more than 10 re-engineered A20FMDV2 variants were synthesized that were cyclized by cysteine perfluoroarylation with DFBP and further modified with D-amino acids and non-proteinogenic analogs to improve the A20FMDV2 peptide's serum stability without sacrificing its affinity and specificity for αvβ6. From this panel, three variants (DFBP-cyclized C2C18 CitTKA_{D}, C2C18 Pₕ R_{D}TKA_{D}, and C1C18 A_{D} R_{D}TK_{D}A_{D}) were identified that persist in serum for ≥24 h and retain binding affinity and specificity to αvβ6⁺ cells, with the first variant demonstrating even better αvβ6 specificity than the original A20FMDV2 peptide. Given these enhanced properties, future in vivo usage of the cyclization and modifications reported herein could greatly improve the bioavailability of the A20FMDV2 peptide for applications concerning cancer diagnosis and treatment.

In addition to the improvements to the A20FMDV2 peptide described above, there are opportunities for further peptide modification and optimization. In the MALDI-ToF MS serum stability studies, N-terminal cleavage of the biotinylated asparagine in modified C2C18 DFBP formulations as well as C-terminal cleavage at or adjacent to threonine was observed for all the cyclized formulations. The N-terminal asparagine and C-terminal threonine can be substituted with D-amino acid analogs to improve A20FMDV2 serum stability without diminishing binding affinity (see, e.g., Hung, K., Harris, P. W. R., Desai, A., Marshall, J. F., and Brimble, M. A. (2017) Structure-activity relationship study of the tumour-targeting peptide A20FMDV2 via modification of Lys16, Leu13, and N- and/or C-terminal functionality. Eur. J. Med. Chem. 136, 154-164). It has further been shown that Lys16 following the RGDLXXL (SEQ ID NO:17) motif can be replaced with 1-ornithine, 1-2,4-diaminobutryic acid, or 1-2,3-diaminopropionic acid and that Leu13 within the RGDLXXL (SEQ ID NO: 17) motif can be replaced with 1-2-aminoisobutryic acid or 1-norvaline without reducing αvβ6 binding. Accordingly, these modifications combined with the cyclization and modifications described above can be implemented to generate additional “super-stable” A20FMDV2 peptide variants.

Another facet of the re-engineered A20FMDV2 variants is their potential for decreased immunogenicity. The inventors' lab has previously shown that repeated doses of PEG-containing micelles loaded with melittin peptides derived from bee venom can cause fatal anaphylactic reactions in immunocompetent mice that are characterized by high titers of serum IgM antibodies that bind PEG (Sylvestre, M., Lv, S., Yang, L. F., Luera, N., Peeler, D. J., Chen, B.-M., Roffler, S. R., and Pun, S. H. (2021) Replacement of L-amino acid peptides with D-amino acid peptides mitigates anti-PEG antibody generation against polymer-peptide conjugates in mice. J. Control. Release. 331, 142-153). Switching to micelles loaded with melittin peptides modified with D-amino acids was found to eliminate this immune hypersensitivity, suggesting that the unmodified melittin peptides served as adjuvants to induce anti-PEG antibody responses. While the A20FMDV2 peptide does not appear to be immunogenic in humans after a single microdose, its derivation from FMDV may raise concerns for human application, especially with PEGylation and after repeated doses. Given all this, the incorporation of D-amino acids and non-proteinogenic analogs into the A20FMDV2 sequence described in this work is likely to also lower the potential immunogenicity of this virus-derived peptide, further increasing its safety profile for clinical applications.

### Experimental Procedures

### Materials

Solvents, including N,N-dimethylformamide (DMF), dichloromethane (DCM), trifluoroacetic acid (TFA), ethyl ether anhydrous, and acetonitrile (ACN), were purchased from Fisher Scientific (Waltham, MA). Standard protected 1-amino acids, D(+)-Biotin, Fmoc-d-Ala-OH, Fmoc-d-Lys(Boc)-OH, Fmoc-d-Arg(Pbf)-OH, Fmoc-Cit-OH, Fmoc-1-Hyp(tBu)-OH, Fmoc-1-Phe(4-Boc-amino)-OH, Fmoc-1-Phe(4-Boc2-guanidino)-OH, Rink Amide resin, ethyl (hydroxyamino)cyanoacetate (Oxyma), N,N′-diisopropylcarbodiimide (DIC), piperidine, and tris-(carboxyethyl)phosphine hydrochloride (TCEP) were purchased from Novabiochem (Darmstadt, Germany), AnaSpec (Fremont, CA), Chem-Impex International (Wood Dale, IL), and Sigma-Aldrich (St. Louis, MO). 1,3-dimethoxybenzene (DMB), triisopropylsilane (TIPS), 1,2-ethanedithiol (EDT), and decafluorobiphenyl (DFBP) were purchased from Acros Organics (Fair Lawn, NJ). The QuantTag Biotin Quantification Kit was purchased from Vector Laboratories (Burlingame, CA). RPMI and DPBS with magnesium and calcium were purchased from Corning (Corning, NY). DMEM, fetal bovine serum (FBS), and StemPro Accutase were purchased from Life Technologies (Carlsbad, CA). Zombie Violet and Streptavidin Alexa Fluor 647 were purchased from BioLegend (San Diego, CA). Bovine serum albumin was purchased from Miltenyi Biotec (Bergisch Gladbach, Germany). 4% paraformaldehyde was purchased from Alfa Aesar (Haverhill, MA). Normal mouse serum for stability studies was prepared in-house. Briefly, mouse whole blood was collected by cardiac puncture into BD Microtainer SST tubes (Becton Dickinson, Franklin Lanes, NJ) and allowed to clot for 30 min. Tubes were then centrifuged according to the manufacturer's instructions and serum was collected and stored at −20° C. until needed. All animal handling protocols were approved by the University of Washington Institutional Animal Care and Use Committee.

### Peptide Synthesis

Sequences of synthesized peptides are listed in Table 1 shown below.

TABLE 1

| | | Weight | SEQ |
|---|---|---|---|
| Peptide | Sequence | (g/mol) | ID No. |
| A20FMDV2 | biotin-NAVPNLRGDLQVLAQKVARTK-amide | 2517.0 | 1 |
| C1C18 DFBP | biotin-CAVPNLRGDLQVLAQKVCRTK-amide | 2832.2 | 2 |
| C1C19 DFBP | biotin-CAVPNLRGDLQVLAQKVACTK-amide | 2747.1 | 3 |
| C1C20 DFBP | biotin-CAVPNLRGDLQVLAQKVARCK-amide | 2802.1 | 4 |
| C2C18 DFBP | biotin-NCVPNLRGDLQVLAQKVCRTK-amide | 2875.2 | 5 |
| C2C19 DFBP | biotin-NCVPNLRGDLQVLAQKVACTK-amide | 2790.1 | 6 |
| C6C17 DFBP | biotin-NAVPNCRGDLQVLAQKCARTK-amide | 2805.1 | 7 |
| C1C18 S-S | biotin-CAVPNLRGDLQVLAQKVCRTK-amide | 2536.1 | 8 |
| C2C18 R_{D}TKA_{D} DFBP | biotin-NCVPNLRGDLQVLAQKVCR_{D}TKA_{D}-amide | 2946.3 | 9 |
| C2C18 CitTKA_{D} DFBP | biotin-NCVPNLRGDLQVLAQKVCCitTKA_{D}-amide | 2947.3 | 10 |
| C2C18 Pₕ R_{D}TKA_{D} DFBP | biotin-NCVPₕNLRGDLQVLAQKVCR_{D}TKA_{D}-amide | 2962.3 | 11 |
| C1C18 A_{D} R_{D}TKDA_{D} DFBP | biotin-CA_{D}VPNLRGDLQVLA_{D}QKVCR_{D}TK_{D}A_{D}-amide | 2903.2 | 12 |
| C2C18 _{A}FGD RTKA_{D} DFBP | biotin-NCVPNL_{A}FGDLQVLAQKVCRTKA_{D}-amide | 2952.3 | 13 |
| C2C18 CitGD DFBP | biotin-NCVPNLCitGDLQVLAQKVCRTK-amide | 2876.2 | 14 |
| C2C18 _{G}FGD R_{D}TKA_{D} DFBP | biotin-NCVPNL_{G}FGDLQVLAQKVCR_{D}TKA_{D}-amide | 2994.3 | 15 |
| C2C18 Pₕ A_{D} R_{D}TKA_{D} DFBP | acetyl-NCVPₕNLRGDLQVLA_{D}QKVCR_{D}TK_{D}-biotin | | 16 |
| | acetyl-NCVPₕNLRGDLQVLA_{D}QKVCR_{D}TK_{D}(Cy5) | | |

C, DFBP-cyclized; C, disulfide-cyclized; R_{D}, D-arginine; A_{D}, D-alanine; Cit, Citrulline; Pₕ, hydroxyproline; K_{D}, D-lysine; _{A}F, 4-aminophenylalanine; _{G}F, 4-guanidinophenylalanine; Cy5, cyanine 5.

Peptide synthesis was performed with a Liberty Blue HT12 automated microwave peptide synthesizer (CEM, Matthews, NC) by Fmoc solid-phase peptide synthesis on a Rink Amide resin support at 0.1 mmol scale. The resin was swelled in 50:50 (v/v) DMF:DCM for 20 min prior to synthesis. Deprotection of Fmoc groups occurred in 20% piperidine in DMF at 90° C. for 65 sec followed by three washes with DMF. Carbodiimide chemistry was used to activate amino acids (AA) at a molar ratio of 1:2:1 AA:DIC:Oxyma in DMF and couplings occurred at 90° C. for 4 min, with the exception for the first amino acid in the synthesis process which was allowed to couple to the resin for 10 min. Double couplings were used for Fmoc-Arg(Pbf)-OH and Fmoc-d-Arg(Pbf)-OH due to the large Pbf group that sterically hinders coupling of these amino acids. After synthesis, side-chain deprotection and cleavage of the peptides from the resin was carried out in 89:5:2.5:2.5:1 (v/v) TFA:DMB:TIPS:EDT:H₂O for 4 h at room temperature with end-over-end mixing. EDT was included in the cleavage cocktail only for cysteine-containing peptides. Cleaved peptides were then double precipitated in cold ethyl ether anhydrous and pelleted at 4500×g for 5 min at 4° C. after each precipitation before drying overnight under vacuum. A small amount of cleaved product was retained and analyzed by MALDI-ToF MS (Bruker AutoFlex II, Billerica, MA) to confirm proper cleavage and deprotection of each peptide. For some of the D-arginine containing peptides, a second 1 h cleavage of the precipitated crude peptide was required due to challenges with removing the Pbf protecting group on d-arginine. Those cleavage reactions were similarly precipitated in ether before being dried overnight. The next day, crude peptides were dissolved in methanol and re-precipitated in ether to wash away remaining protecting groups. Finally, the crude peptide was dried again overnight under vacuum before HPLC purification.

### Reverse-Phase HPLC Purification

Crude peptides were resuspended at 80 mg/mL in 20% ACN in H₂O containing 0.1% TFA (v/v), syringe filtered, and purified to >95% purity by reverse-phase HPLC on an Agilent 1260 Infinity (Santa Clara, CA) equipped with a Synergi 4u Fusion-RP C18 semipreparative column (Phenomenex). Monitoring 220 nm absorbance, a flow rate of 5 mL/min and a 20-65% 8-min linear solvent gradient of ACN in H₂O with 0.1% TFA were used for purification of these peptides. The molecular weights of purified peptides were confirmed by MALDI-ToF MS and were consistently within 1-2 g/mol of the expected values. After purification, organic solvent was removed by rotary evaporation and peptides were lyophilized and stored at −20° C. until further usage.

### Peptide Cyclization by Perfluoroarylation

In-solution cyclization of peptides with DFBP was conducted as previously described (Ngambenjawong, C., Pineda, J. M. B., and Pun, S. H. (2016) Engineering an Affinity-Enhanced Peptide through Optimization of Cyclization Chemistry. Bioconjug. Chem. 27, 2854-2862), but with some modifications. Briefly, 20 mg purified linear peptide was dissolved in 3 mL DMF with 2 molar equivalents of each TCEP and DFBP. Then, 1.5 mL 50 mM tris base in DMF was freshly prepared and added to the reaction before vortexing (4.5 mL total). After an overnight incubation at room temperature with end-over-end mixing, the cyclization reaction was diluted in 10 mL H₂O containing 0.1% TFA and peptide was desalted with a Sep-Pak C18 cartridge (Waters, Milford, MA). Peptide was eluted from the cartridge in 50:50 (v/v) ACN:H₂O containing 0.1% TFA and proper cyclization was confirmed by MALDI-ToF MS. Organic solvent was subsequently removed by rotary evaporation and cyclized peptides were lyophilized and stored at −20° C. until further usage.

### Cell Line Culture

The K562 and A375P cell lines used in binding studies were purchased from ATCC. The K562 αvβ6:mCherry cell line was a kind gift from Audrey Olshefsky (Pun and King Labs, University of Washington) and was generated by nucleofection of K562 cells with two linear DNA fragments, one encoding the αv integrin and a puromycin resistance gene and the other encoding the β6 integrin and a fluorescent mCherry reporter. After nucleofection, K562 αvβ6:mCherry cells were purified by puromycin selection and FACS sorting. K562 and K562 αvβ6:mCherry suspension cells were cultured in RPMI 1640 medium with 1-glutamine and 10% FBS. Adherent A375P cells were cultured in high-glucose DMEM medium with 1-glutamine and 10% FBS and were detached with StemPro Accutase prior to flow cytometry binding studies to preserve extracellular integrin expression.

### Flow Cytometry Binding Studies

Biotinylated peptide stocks were prepared in H₂O at approximately 5 mM and the exact concentration was measured using a QuantTag Biotin Quantification Kit. Stocks were stored at 4° C. and used for binding studies within two weeks of preparation. Prior to binding, cells were pre-stained with Zombie Violet in DPBS (0.2 μL per 100 μL per 10⁶ cells) for 15 min at room temperature for dead cell discrimination. Meanwhile, peptide stocks were serially diluted in DPBS with calcium and magnesium over ice. Cells were then washed at 4° C. with DPBS 1% BSA to neutralize the Zombie Violet, plated in a U-bottom black 96-well plate (2×10⁵ per well) over ice, and stained with 100 μL peptide solutions for 20 min at 4° C. Cells were then washed twice with 200 μL cold DPBS and incubated with 100 μL streptavidin Alexa Fluor 647 in DPBS (1:500) for 20 min at 4° C. Cells were subsequently washed twice as before and resuspended in 200 μL DPBS 0.1% PFA for assaying on an Attune NxT Flow Cytometer (Life Technologies, Carlsbad, CA). Data was analyzed by FlowJo v10 software (Becton Dickinson, Ashland, OR) and median fluorescence intensity of singlet live cell events were used as measurements of binding. Data was normalized to A20FMDV2 binding prior to generating binding curves, apparent K_{D} values, bar graphs, and statistical testing in GraphPad Prism 6 software (San Diego, CA).

### MALDI ToF MS Serum Stability

Peptides were incubated and extracted from serum as previously described (Ngambenjawong, C., Pineda, J. M. B., and Pun, S. H. (2016) Engineering an Affinity-Enhanced Peptide through Optimization of Cyclization Chemistry. Bioconjug. Chem. 27, 2854-2862; Sellers, D. L., Tan, J.-K. Y., Pineda, J. M. B., Peeler, D. J., Porubsky, V. L., Olden, B. R., Salipante, S. J., and Pun, S. H. (2019) Targeting Ligands Deliver Model Drug Cargo into the Central Nervous System along Autonomic Neurons. ACS Nano. 13, 10961-10971), but with some adjustments to limit serum protein carryover during extractions. Briefly, peptide stocks were diluted to 10 mg/mL in H₂O and subsequently diluted 1:10 (v/v) in normal mouse serum for incubation at 37° C. in an incubator. At specified timepoints, 40 μL of the peptide/serum mixture was removed and precipitated in an equal volume of cold ACN. Precipitated serum proteins were pelleted by centrifugation at 15000×g for 5 min and the supernatant with peptides (80 μL) was collected. To extract any remaining peptides, 80 μL cold 1:3 (v/v) H₂O:ACN was added to the pellet, sonicated for 10 min, and centrifuged as before. The resulting supernatant was combined with the old one and dried under vacuum on a Savant ISS110 SpeedVac Concentrator (Thermo Fisher, Waltham, MA). The peptide pellet was then resuspended in 50 μL H₂O and sonicated for 10 min. A small aliquot of resuspended peptide was further diluted 1:10 (v/v) in H₂O and analyzed by MALDI-ToF MS. Resulting mass spectrums at the different timepoints were aligned and plotted in FlexAnalysis software (Bruker, Billerica, MA). Predicted sequences of degradation products based on product molecular weight were determined using a custom Java program found on the peptide-serum-stability tool called stability.jar (Sellers, D. L., Tan, J.-K. Y., Pineda, J. M. B., Peeler, D. J., Porubsky, V. L., Olden, B. R., Salipante, S. J., and Pun, S. H. (2019) Targeting Ligands Deliver Model Drug Cargo into the Central Nervous System along Autonomic Neurons. ACS Nano. 13, 10961-10971).

### LC-MS Serum Stability

Remaining extracted peptides from MALDI ToF MS serum stability studies were dried as before and stored as dried pellets at room temperature. Within a day of LC-MS runs, pellets were resuspended in 100 μL 95:5 (v/v) H₂O:ACN, sonicated for 10 min, and submitted for LC-MS analysis at the University of Washington School of Pharmacy Mass Spectrometry Center. Samples were injected on a TripleTOF 5600+ instrument (AB Sciex, Framingham, MA) equipped with a 2.1 mm×50 mm BEH C18 column (Waters, Milford, MA). Peptides were separated over a 5-100% 4-min linear solvent gradient of ACN in H₂O with 0.1% formic acid, and peak area corresponding to the peptide molecular weight was used for quantification of remaining intact peptide. Peak areas across different timepoints were normalized to that of a 0 h control sample that was extracted immediately after mixing the peptide with the serum. Exponential decay curves were generated in GraphPad Prism 6 software.

### Example 2

This Example describes the design of a cyclized A20FMDV2 variant with selective amino acid modifications and evaluation of its rate of internalization in BxPC-3 cells. This Example demonstrates that DFBP-cyclized C2C18 PₕA_{D}R_{D}TK_{D} (SEQ ID NO:16) was rapidly internalized by BxPC-3 cells at 37° C. with a cell surface half-life of just 11.93 minutes as further described below.

### Results

### Modified Decafluorobiphenyl (DFBP)-Cyclized A20FMDV2 Peptide is Rapidly Internalized by αvβ6⁺ Pancreatic BxPC-3 Cells

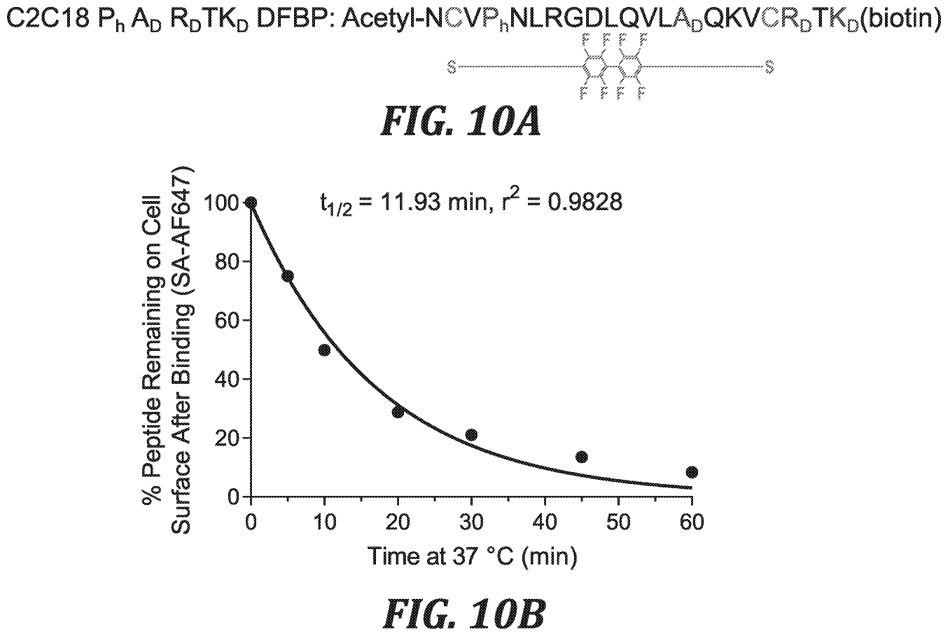

Targeted peptide internalization is important for theranostic applications. Therapeutically, internalization of peptides by target cells allows delivery of associated drug cargos to their site of action, typically in the cytosol or nucleus. For diagnostics, the same process can enable prolonged retention of associated labels/dyes at sites of interest, increasing the window for imaging and potentially improving the signal-to-noise ratio. Accordingly, the rate of internalization of DFBP-cyclized C2C18 PₕA_{D}R_{D}TK_{D} (P4Pₕ; A14A_{D}; R19R_{D}; K21K_{D}) (SEQ ID NO:16) was evaluated. This modified peptide combines the amino acid modifications that were previously determined to improve peptide serum stability while retaining the αvβ6 specificity of the original A20FMDV2 peptide (FIG. 10A). As a relevant theranostic target, pancreatic adenocarcinoma BxPC-3 cells were selected, which endogenously express αvβ6 at moderate levels. To assess internalization, BxPC-3 cells were labeled with biotinylated peptide at 4° C. to saturate extracellular αvβ6 receptors, washed to remove excess peptide, and then incubated at 37° C. for varying times up to 60 min to induce internalization. Cells were then washed and extracellularly stained with streptavidin Alexa Fluor 647 at 4° C. to quantitatively measure the loss of surface-bound peptide (i.e. internalization) by flow cytometry. As shown, C2C18 Pₕ A_{D} R_{D}TK_{D} DFBP (SEQ ID NO:16) was rapidly internalized by BxPC-3 cells at 37° C. with a cell surface half-life of just 11.93 min (FIG. 10B). Given this data, one of skill in the art would recognize and predict that the engineered A20FMDV2 peptides disclosed in this application can be feasibly implemented in drug delivery and imaging applications.

### Methods

### Peptide Synthesis, Purification, and Cyclization

The biotinylated C2C18 PₕA_{D}R_{D}TK_{D} DFBP peptide (SEQ ID NO:16) was synthesized as previously described (see Example 1), but with minor modifications during synthesis to enable biotinylation of the D-lysine side-chain amino group at the peptide C-terminus. Briefly, Fmoc-D-Lys(Mtt)-OH (Alfa Aesar) was used in addition to other standard protected L- and D-amino acids to synthesize peptide on resin with an acid-labile methyltrityl (Mtt) protecting the D-lysine side-chain amino group. On-resin acetylation of the peptide N-terminus was then performed twice in 10 mL 3:2:1 (v/v) DCM:pyridine:acetic anhydride for 1 h at room temperature with end-over-end mixing. After confirming N-acetylation by a Kaiser Test, the Mtt-protecting group on D-Lys(Mtt) at the peptide C-terminus was selectively removed by repeatedly incubating the resin with 10 mL 2% TFA in DCM (v/v) for 15 min at room temperature with end-over-end mixing until the deprotection solution turned from yellow-orange to clear (5-10 times). Resin was then thoroughly washed with each DCM, DMF, and methanol before successful Mtt-deprotection was qualitatively confirmed by Kaiser Test. The resin with N-acetylated and Mtt-deprotected peptide was swelled in 50:50 (v/v) DMF:DCM for 20 min prior to biotinylation on the Liberty Blue HT12 automated microwave peptide synthesizer (CEM, Matthews, NC). Subsequent cleavage of peptide from the resin and downstream reverse-phase HPLC purification and cyclization was performed as previously described (see Example 1).

### Cell Line Culture

The BxPC-3 cell line was purchased from ATCC and cultured in RPMI 1640 medium with L-glutamine (Corning) and 10% FBS (Life Technologies). Cells were detached with 0.25% Trypsin-EDTA prior to flow cytometry studies, which was not found to affect engineered peptide binding to αvβ6 on the cells (data not shown).

### Flow Cytometry Internalization Study

Biotinylated DFBP-cyclized C2C18 PₕA_{D}R_{D}TK_{D} (SEQ ID NO:16) was prepared in H₂O at approximately 5 mM and the exact concentration was measured using a QuantTag Biotin Quantification Kit. The stock was stored at 4° C. and used for the internalization study within two weeks of preparation. Prior to binding, trypsin-lifted BxPC-3 cells were pre-stained with Zombie Violet in DPBS (0.2 μL per 100 μL per 10⁶ cells) for 15 min at room temperature for dead cell discrimination. Meanwhile, the peptide stock was diluted to 400 nM in DPBS with calcium and magnesium over ice. Cells were then washed at 4° C. with DPBS 1% BSA to neutralize the Zombie Violet, plated in a U-bottom 96-well plate (2×10⁵ per well) over ice, and stained with 100 μL 400 nM peptide solution for 20 min at 4° C. Cells were then washed twice with 200 μL cold DPBS, resuspended in complete media on ice, and transferred to a 37° C. incubator at different times over a 60-min period to induce internalization. Afterwards, the cells were transferred back on ice to stop further internalization, washed twice with 200 μL cold DPBS to remove media, and incubated with 100 μL streptavidin Alexa Fluor 647 in DPBS (1:500) for 20 min at 4° C. Cells were subsequently washed twice as before and resuspended in 200 μL DPBS 0.1% PFA for assaying on an Attune NxT Flow Cytometer (Life Technologies, Carlsbad, CA). Data was analyzed by FlowJo v10 software (Becton Dickinson, Ashland, OR) and median fluorescence intensity of singlet live cell events were used as measurements of remaining peptide on the cell surface. Data was normalized to peptide binding on cells without 37° C. incubation (no internalization control) prior to generating exponential decay curves in GraphPad Prism 6 software (San Diego, CA).

### Example 3

This Example describes the design of a cyclized A20FMDV2 variant with selective amino acid modifications and evaluation of its circulation half-life in mice. This Example demonstrates that Cy5-labeled C2C18 PₕA_{D}R_{D}TK_{D} DFBP (SEQ ID NO:16) displayed a short distribution half-life of ˜2 min and a longer elimination half-life of ˜50 min, consistent with a two-compartment pharmacokinetic model.

### Results

### Modified Decafluorobiphenyl (DFBP)-Cyclized A20FMDV2 Peptide (SEQ ID NO:16) Exhibits Moderate Plasma Circulation Half-Life in Mice

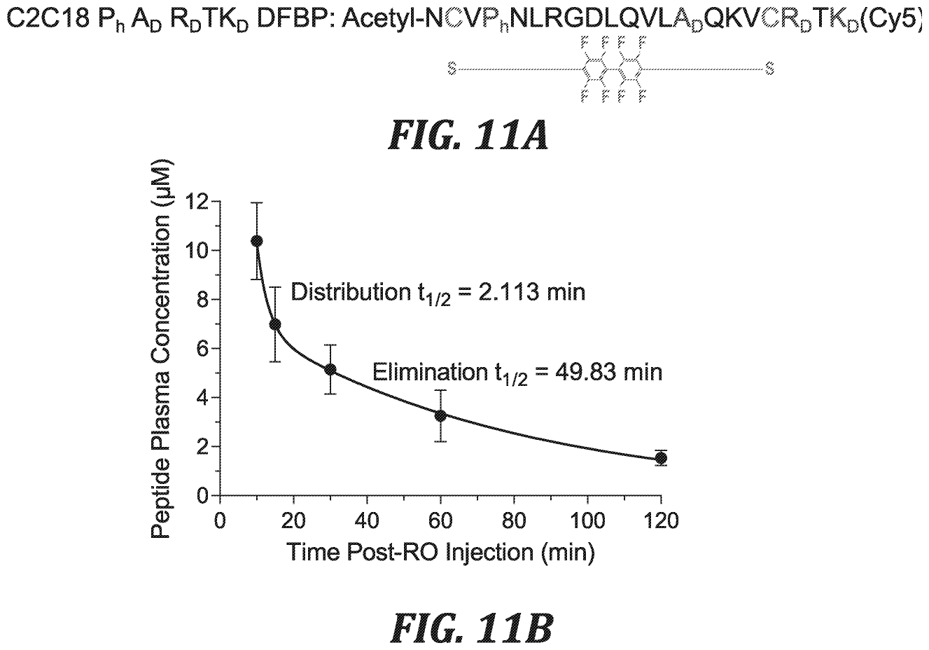

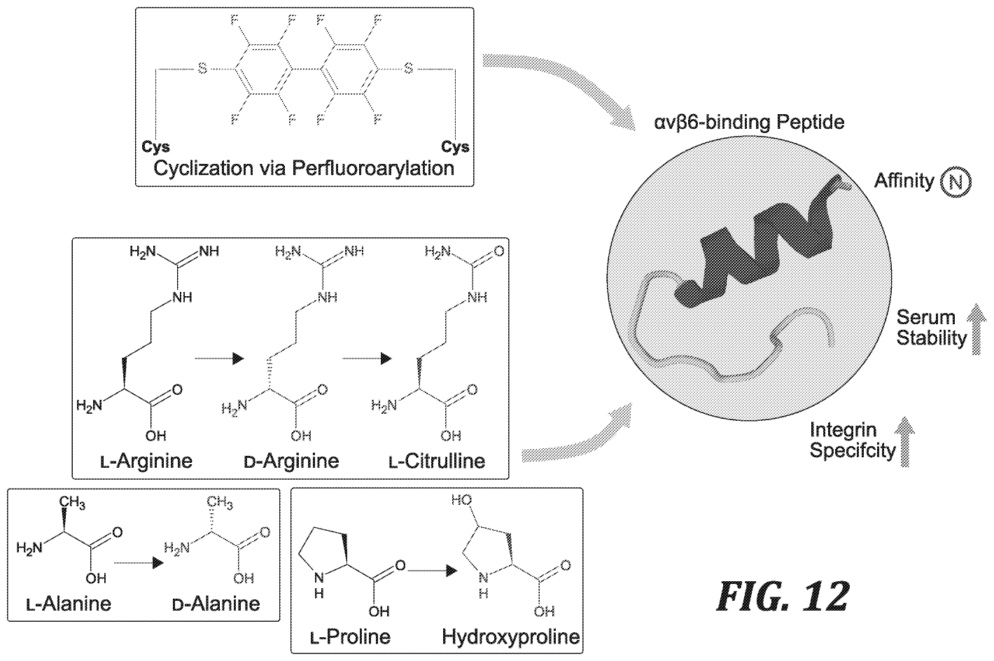

The short circulation half-life of peptides in vivo poses a major challenge for their usage in theranostic applications. A peptide's circulation half-life should be sufficiently long for effective targeting, but at the same time it should be sufficiently short to achieve high signal-to-noise quickly. Proteolytic degradation and renal clearance ultimately dictate the circulation half-life of peptides in vivo. In Example 1, DFBP cyclization and amino acid substitutions/modifications were used to enhance the proteolytic stability of the A20FMDV2 peptide, achieving long in vitro serum half-lives of 4.5-6.6 h. As these engineered improvements to A20FMDV2 effectively eliminated proteolytic cleavage as a significant contributor to in vivo removal of these peptide, this Example discloses characterization of the circulation half-life of the engineered A20FMDV2 peptide in mice. For fluorescence detection of peptide in plasma, DFBP-cyclized C2C18 PₕA_{D}R_{D}TK_{D} (P4Pₕ; A14A_{D}; R19R_{D}; K21K_{D}) (SEQ ID NO:16) was labeled with Cy5 at the C-terminal D-lysine via NHS chemistry (FIG. 11A). After retro-orbital injection in NOD scid mice, Cy5-labeled C2C18 PₕA_{D}R_{D}TK_{D} DFBP (SEQ ID NO:16) displayed a short distribution half-life of ˜2 min and a longer elimination half-life of ˜50 min, consistent with a two-compartment pharmacokinetic model. This elimination half-life is comparable to that of octreotide (t_{1/2} ˜90 min), a successful peptide-based radiopharmaceutical used in the diagnosis and treatment of neuroendocrine tumors (Harris, A. G. (1994) Somatostatin and somatostatin analogues: pharmacokinetics and pharmacodynamic effects. Gut. 35, S1-S4), demonstrating the potential of engineered A20FMDV2 peptides for cancer therapy and imaging.

### Methods

### Peptide Synthesis, Purification, and Cyclization

The Cy5-labeled C2C18 PₕA_{D}R_{D}TK_{D} DFBP peptide (SEQ ID NO:16) was synthesized as previously described (see Example 1), but with minor modifications during synthesis to enable Cy5 labeling of the D-lysine side-chain amino group at the peptide C-terminus. Briefly, Fmoc-D-Lys(Mtt)-OH (Alfa Aesar) was used in addition to other standard protected L- and D-amino acids to synthesize peptide on resin with an acid-labile methyltrityl (Mtt) protecting the D-lysine side-chain amino group. On-resin acetylation of the peptide N-terminus was then performed twice in 10 mL 3:2:1 (v/v) DCM:pyridine:acetic anhydride for 1 h at room temperature with end-over-end mixing. After confirming N-acetylation by a Kaiser Test, the Mtt-protecting group on D-Lys(Mtt) at the peptide C-terminus was selectively removed by repeatedly incubating the resin with 10 mL 2% TFA in DCM (v/v) for 15 min at room temperature with end-over-end mixing until the deprotection solution turned from yellow-orange to clear (5-10 times). Resin was then thoroughly washed with each DCM, DMF, and methanol before successful Mtt-deprotection was qualitatively confirmed by Kaiser Test. For on-resin coupling of NHS-Cy5 (BroadPharm), resin with N-acetylated and Mtt-deprotected peptide was swelled in DCM and then exchanged to 5 mL DCM with 2 molar equivalents NHS-Cy5, 3 molar equivalents EDC, and 8 molar equivalents of triethylamine. After an overnight incubation at room temperature with end-over-end mixing, the resin with Cy5-coupled peptide was washed 3 times in DCM before cleavage. Subsequent cleavage of peptide from the resin and downstream reverse-phase HPLC purification and cyclization was performed as previously described (see Example 1).

### In Vivo Circulation Half-Life

Cy5-labeled C2C18 PₕA_{D}R_{D}TK_{D} DFBP (SEQ ID NO:16) (10 nmol) in DPBS was injected into four 8-12-week-old NOD scid mice via retro-orbital route. Blood was drawn at 10-, 15-, 30-, 60-, and 120-min post-injection and collected into pre-weighed tubes with 50 μL DPBS 5 mM EDTA. After blood collection, the tubes were re-weighed to determine the volume of blood collected. The diluted blood was then centrifuged at 1000×g for 10 min at 4° C., and 50 μL of the supernatant was measured for Cy5 fluorescence (Ex: 633/9 nm; Em: 670/20 nm) using an Infinite 200 PRO plate reader (Tecan). The measured fluorescence values at each timepoint were compared to a standard curve of Cy5-labeled C2C18 PₕA_{D}R_{D}TK_{D} DFBP (SEQ ID NO:16) and normalized by their respective dilution factors to estimate the peptide concentration in undiluted plasma. Data from samples with less than 1 μL collected blood (two mice at 10-min post-injection and one mouse at 15-min post-injection) were excluded prior to generating two-phase exponential decay curves in GraphPad Prism 6 software (San Diego, CA).

### Example 4

This Example describes the use of engineered A20FMDV2 peptides for selective in vivo targeting of αvβ6⁺ expressing tumors.

To demonstrate selective in vivo targeting of αvβ6⁺ tumors, the engineered A20FMDV2 peptides described in the Examples section will be administered to two different bilateral xenograft tumor models. Specifically, NOD scid or similarly immunocompromised mice will be subcutaneously injected with K562 and K562 αvβ6:mCherry cells or A375P (αv⁻β6⁺) and BxPC-3 (αv⁺β6⁺) cells in their left and right flanks, respectively. After giving tumors sufficient time to reach a palpable size (9-28 days, 70-200 mm³), tag-labeled (e.g. FITC, Cy5, HRP) engineered peptides will be injected intravenously into the mice via the retro-orbital sinus. Tag alone or saline will be also injected as a control arm. Peptide biodistribution and tumor targeting will be characterized by up to three different methods: qualitative fluorescent Xenogen imaging of live animals, semi-quantitative fluorescent Xenogen imaging of harvested organs and tumors, and quantitative plate reader measurements of peptide signal in dissociated organs and tumors. Organ and tumor weights and their protein concentration after dissociation will also be determined to normalize for differences in sample size. Due to the increased serum stability and increased specificity of these engineered peptide, it is expected that the engineered peptides will selectively gather in the right αv⁺β6⁺ tumors over the left αv⁻β6⁻ or αv⁺β6⁻ tumors, as evidenced by higher tag signal per tumor weight or protein content. Additionally, it is expected that some of the engineered peptides will accumulate in the kidneys and liver of the mice due to normal renal and metabolic clearance.

While illustrative embodiments have been illustrated and described, it will be appreciated that various changes can be made therein without departing from the spirit and scope of the invention.

1. A synthetic peptide comprising an amino acid sequence of X₁X₂VX₃NLRGDLQVLX₄QKVCX₅T (SEQ ID NO: 18), wherein X₁, X₂, or both X₁ and X₂ is a cysteine.

2. The synthetic peptide of claim 1 comprising an amino acid sequence of X₁X₂VX₃NLRGDLQVLX₄QKVCX₅TX₆ (SEQ ID NO:19), wherein X₁, X₂, or both X₁ and X₂ is a cysteine.

3. The synthetic peptide of claim 2 comprising an amino acid sequence of X₁X₂VX₃NLRGDLQVLX₄QKVCX₅TX₆X₇ (SEQ ID NO:20), wherein X₁, X₂, or both X₁ and X₂ is a cysteine.

4. The synthetic peptide of claim 3, wherein X₃, X₄, X₅, X₆, X₇, or any combination thereof, is a non-natural amino acid.

5. The synthetic peptide of claim 1, comprising a cysteine at X₁, wherein the synthetic peptide has a cyclic portion at X₁ and a second cysteine, and wherein the synthetic peptide has at least one perfluoroaryl compound linker at a thiol group of X₁ and a thiol group of the second cysteine.

6. The synthetic peptide of claim 1, comprising a cysteine at X₂, wherein the synthetic peptide has a cyclic portion at X₂ and a second cysteine, and wherein the synthetic peptide has at least one perfluoroaryl compound linker at a thiol group of X₂ and a thiol group of the second cysteine.

7. The synthetic peptide of claim 1, wherein X₃, X₄, X₅, X₆, X₇, or any combination thereof, is a non-natural amino acid independently selected from the group consisting of: a hydroxyproline, a D-arginine, a D-alanine, a citrulline, and a D-lysine.

8. The synthetic peptide of claim 1, wherein X₅ is a non-natural amino acid.

9. The synthetic peptide of claim 8, wherein X₅ is D-arginine or citrulline.

10. (canceled)

11. The synthetic peptide of claim 6, wherein the synthetic peptide comprises an amino acid sequence of X₁X₂VX₃NLRGDLQVLX₄QKVCX₅TX₆ (SEQ ID NO:19).

12. The synthetic peptide of claim 11, wherein X₁ is an asparagine, X₃ is a hydroxyproline, X₄ is a D-alanine, X₅ is a D-arginine, and X₆ is a D-lysine.

13. The synthetic peptide of claim 11, further comprising a therapeutic agent or an imaging agent bound to X₆.

14. (canceled)

15. The synthetic peptide of claim 6, wherein the synthetic peptide comprises an amino acid sequence of X₁X₂VX₃NLRGDLQVLX₄QKVCX₅TX₆X₇ (SEQ ID NO:20).

16. The synthetic peptide of claim 15, wherein X₁ is an asparagine, X₃ is a proline or a hydroxyproline, X₄ is an alanine, X₅ is a D-arginine or a citrulline, X₆ is a lysine, and X₇ is a D-alanine.

17. The synthetic peptide of claim 15, wherein X₁ is an asparagine X₃ is a proline or a hydroxyproline X₄ is an alanine, X₅ is a citrulline or a D-alanine, X₆ is a lysine, and X₇ is a D-alanine.

18. (canceled)

19. The synthetic peptide of claim 5, wherein the synthetic peptide comprises an amino acid sequence of X₁X₂VX₃NLRGDLQVLX₄QKVCX₅TX₆X₇ (SEQ ID NO:20).

20. The synthetic peptide of claim 19, wherein X₂ is a D-alanine, X₃ is a proline, X₄ is a D-alanine, X₅ is a D-arginine, X₆ is a D-lysine, and X₇ is a D-alanine.

21. The synthetic peptide of claim 19, further comprising a therapeutic agent or an imaging agent bound to X₁.

22. (canceled)

23. (canceled)

24. A cyclized peptide that specifically binds to integrin αvϑ6, the peptide comprising a sequence as set forth in one of SEQ ID NOs: 2-7.

25.-29. (canceled)

30. A method of inhibiting growth of a cancer cell overexpressing integrin αvϑ6, the method comprising administering to a patient in need thereof a composition comprising a synthetic peptide that specifically binds αvϑ6 and a pharmaceutically acceptable carrier, the synthetic peptide comprising an amino acid sequence selected from the group of X₁X₂VX₃NLRGDLQVLX₄QKVCX₅T (SEQ ID NO:18), X₁X₂VX₃NLRGDLQVLX₄QKVCX₅TX₆ (SEQ ID NO:19), and X₁X₂VX₃NLRGDLQVLX₄QKVCX₅TX₆X₇ (SEQ ID NO:20), wherein X₁, X₂, or both X₁ and X₂ is a cysteine.

31.-46. (canceled)
